(19) United States
(12) Patent Application Publication
(10) Pub. No.: US 2009/0062237 A1
(43) Pub. Date: Mar. 5, 2009

(54) EVALUATING IMMUNE COMPETENCE

(75) Inventors: Roshini Sarah Abraham, Rochester, MN (US); Vijay Nandakumar, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 12/139,115

(22) Filed: Jun. 13, 2008

Related U.S. Application Data
(60) Provisional application No. 60/944,241, filed on Jun. 15, 2007.

(51) Int. Cl. A61K 31/675; C07K 7/00; C12Q 1/70; A61P 31/00; A61K 31/445

(52) U.S. Cl. 514/86; 530/328; 435/5; 514/326

(57) ABSTRACT

This document relates to methods and materials involved in evaluating immune competence in a subject. For example, methods and materials for evaluating immune competence in a subject to BK virus (BKV) are provided. Also provided are isolated peptides, compositions, and methods useful for evaluating whether or not a subject is BKV immunocompetent. Also provided are methods for treating BK virus nephropathy in a subject that is BKV immune incompetent.

### CROSS-REFERENCE TO RELATED APPLICATIONS

This application claims priority under 35 U.S.C. §119(e) of U.S. provisional application Ser. No. 60/944,241, filed Jun. 15, 2007. The disclosure of the prior application is considered part of (and is incorporated by reference in) the disclosure of this application.

### BACKGROUND

1. Technical Field

This document relates to methods and materials involved in evaluating immune competence to a pathogen, such as BK virus, in a subject (e.g., a human subject).

2. Background Information

The BK virus (BKV) is a human polyoma virus (Papovaviridae), and BKV seroprevalence is almost ubiquitous with over 90% of all people being seropositive by age 10. The majority of primary infections are asymptomatic or minimally symptomatic. After primary infection, a latent infection is established in renal epithelial cells. In states of relative or absolute immunodeficiency (including that caused by immunosuppression), the BKV can reactivate and cause disease. BKV reactivation is most common in renal transplant and bone marrow transplant patients. BKV-associated nephropathy has become increasingly recognized as a significant cause of renal dysfunction and loss of the transplanted kidney in renal transplant patients. Urine BKV DNA load is usually 105 fold higher than serum viral DNA load and may be present without viremia. Therefore, absence of BKV DNA in serum does not indicate a lack of BKV reactivation in renal transplant patients. No studies to date have clearly identified factors which would help predict those at risk for BKV nephropathy.

### SUMMARY

Provided herein are methods and materials related to evaluating immune competence to BK virus. For example, this document provides peptides, compositions, kits, and methods that can be used to evaluate immune competence to BK virus.

In one aspect, this document provides an isolated peptide. The isolated polypeptide can be selected from the group consisting of SEQ ID NOS: 1-107, SEQ ID NOS:109-258, and SEQ ID NOS:260-373.

In another aspect, this document provides a composition comprising an MHC molecule and a peptide. The peptide can be selected from the group consisting of SEQ ID NOS:1-107, SEQ ID NOS:109-258, and SEQ ID NOS:260-373. The MHC molecule can be selected from the group consisting of HLA-B7, HLA-A1, HLA-A2, HLA-B8 and HLA-B35. The peptide can be selected from the group consisting of SEQ ID NO:342, SEQ ID NO:252, SEQ ID NO:2, SEQ ID NO:80, SEQ ID NO:14, and SEQ ID NO:182.

In a further aspect, this document provides a composition comprising an MHC molecule and a peptide. The peptide can be selected from the group consisting of SEQ ID NOS:1-373, where the MHC molecule is selected from the group consisting of HLA-B7 and HLA-A1.

Also provided herein is a composition comprising an MHC/peptide multimer and a peptide. The peptide can be selected from the group consisting of SEQ ID NOS: 1-373. The MHC molecule can be selected from the group consisting of HLA-B7, HLA-A1, and HLA-A2. The MHC molecule can be selected from the group consisting of HLA-B8 and HLA-B35. The peptide can be selected from the group consisting of SEQ ID NO:342, SEQ ID NO:252, SEQ ID NO:2, SEQ ID NO:80, SEQ ID NO:14, SEQ ID NO:182, SEQ ID NO:108, and SEQ ID NO:259. The composition can comprise more than one different peptide. The composition can comprise 2, 3, 4, or more different peptides.

In another aspect, this document provides a kit. The kit comprises a peptide and an antibody. The peptide can be selected from the group consisting of SEQ ID NOS:1-373. The antibody can be selected from the group consisting of anti-interferon-gamma, anti-TNF-alpha, anti-IL-2, anti-CD107a, and anti-CD107b.

Also provided herein is a method of evaluating whether or not a subject is BK Virus immunocompetent. The method comprises contacting CD8+ T cells from said subject with a composition comprising a peptide selected from the group consisting of SEQ ID NOS:1-373, and evaluating one or more of said subject's BK Virus-specific CD8+ T cell parameters as compared to the minimum BK Virus-specific CD8+ T cell parameters required for BK Virus immunocompetence. The peptide can be part of an MHC/peptide complex. The peptide can be part of an MHC/peptide multimer. The evaluating can comprise performing flow cytometry. The evaluating can comprise performing an ELISA-based assay.

In another aspect, this document provides a method of treating BK Virus nephropathy in a transplant patient. The method comprises evaluating whether said patient is BK Virus immunocompetent or immune incompetent, and administering an appropriate dose of an immunosuppressive drug. The immunosuppressive drug can be tacrolimus. The immunosuppressive drug can be rapamycin.

In a further aspect, a method of treating BK Virus nephropathy in a transplant patient is provided. The method comprises determining whether said patient is BK Virus immune incompetent, and based on said determination of immunodeficiency, administering an appropriate dose of an antiviral drug. The antiviral drug can be cidifovir.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a table of estimated and actual affinities of BK polyomavirus protein polypeptides for HLA-B7.

FIGS. 2A-2B are a table of estimated affinities of BK polyomavirus protein polypeptides for HLA-B7.

FIGS. 3A-3M are a table of estimated affinities of BK polyomavirus protein polypeptides for HLA-B7.

FIGS. 4A-4B are a table of estimated and actual affinities of BK polyomavirus protein polypeptides for HLA-A1.

FIGS. 5A-5F are a table of estimated affinities of BK polyomavirus protein polypeptides for HLA-A1.

FIGS. 6A-6L are a table of estimated affinities of BK polyomavirus protein polypeptides for HLA-A1.

FIGS. 7A-7B are a table of estimated affinities of BK polyomavirus protein polypeptides and mutant polypeptides for HLA-B7.

FIGS. 8A-8C are a table of estimated affinities of BK polyomavirus protein polypeptides and mutant polypeptides for HLA-A1.

### DETAILED DESCRIPTION

This document provides methods and materials related to evaluating immune competence in a subject, e.g., a mammal such as a human, a non-human primate, a rodent, a dog, a cat, a bovine, or an equine. An individual's CD8+ T cell immune competence can be determined by assessing the number of BK virus (BKV)-specific CD8+ T cells and their activation potential based on the production of the cytokine, IFN-gamma, and potential cytotoxic function based on CD107a and CD107b as markers of degranulation. The methods described herein allow for a comprehensive assessment of BKV-specific CD8+ T cell immunity and provides a more accurate picture of the nature of BKV reactivation and the corresponding immune response than evaluating viremia alone.

This document provides methods and materials for evaluating CD8+ T cell immune competence in a subject to BKV. Also provided herein are isolated peptides, compositions (e.g., MHC/peptide complexes and MHC/peptide multimers), and methods useful for evaluating whether a subject is BKV immunocompetent or immune incompetent. Also provided are methods for detecting and treating BK Virus nephropathy in a subject that is BKV immune incompetent.

Isolated peptides described herein are BKV MHC Class I epitopes. An isolated peptide can be a portion of any BKV viral polypeptide. Portions of BKV viral capsid polypeptides are particularly useful peptides because they are likely to be accessible to immune system cells. For example, BKV VP1 (GenBank Accession No. AAT47405) is the most abundant capsid polypeptide.

The term “isolated” with respect to a peptide refers to a peptide that has been separated from cellular components with which it is naturally accompanied. Typically, a peptide provided herein is isolated when it is at least 60 percent (e.g., 65, 70, 75, 80, 90, 95, or 99 percent), by weight, free from proteins and naturally-occurring organic molecules with which it is naturally associated. In general, an isolated peptide will yield a single major band on a non-reducing polyacrylamide gel. An isolated peptide can be, without limitation, a peptide produced recombinantly from a DNA molecule, a peptide purified from a cell, or a chemically synthesized peptide. In some cases, an isolated peptide can be a peptide preparation that contains one of the peptides set forth in SEQ ID NOS:1-373.

Peptides of the invention can include amino acids which may be natural or unnatural and in the D or L form, and can be synthesized according to standard synthesis methods such as those disclosed in, e.g., WO 01/09188 and WO 01/08712. In some embodiments, only natural amino acids are employed. In some cases, only amino acids in the L form are used, or only amino acids in the D-form. For suitability as MHC Class I epitopes, it is preferred that the peptides have between 5 to 20 amino acids (e.g., between 7 and 15 amino acids, or between 8 and 11 amino acids (e.g., 8, 9, 10, or 11 amino acids in length)). Amino acids with many different protecting groups appropriate for immediate use in the solid phase synthesis of peptides are commercially available.

A peptide can be identified as an MHC Class I epitope by determining whether the peptide has binding affinity for an MHC Class I molecule. Peptides can be assayed for affinity to the appropriate MHC Class I molecules using standard methods. For example, peptides can be screened for binding to an HLA molecule by displacement of labeled reference peptides bound to an HLA molecule. See, e.g., the methods described in the Examples below. An MHC Class I molecule can be encoded by any MHC Class I allele. Non-limiting examples of MIC Class I molecules encoded by MHC Class I alleles include HLA-A1, -A2, -B7, -B8 and -B35.

Suitable peptides can be determined to have log IC₅₀ (i.e., log[(nM)] for an MHC Class I molecule that is less than 6.0 (e.g., a log IC₅₀ that is less than 5.5, or a log IC₅₀ that is less than 4.7). Suitable peptides include the amino acid sequences set forth in SEQ ID NOS:1-355. Other suitable peptides can be a portion of a BKV viral polypeptide that contains a mutation. A BKV viral polypeptide can contain a mutation such as a substitution, deletion, or insertion of one or more amino acids (e.g., 1, 2, 3, or 4 amino acids). Peptides from BKV polypeptides having a mutation include SEQ ID NOS:356-373.

Peptides described herein can be incorporated into compositions of matter. Such compositions can include MHC/peptide complexes. MHC/peptide complexes can be made using standard procedures. For example, an MHC/peptide complex can be produced by recombinantly synthesizing an MHC heavy chain molecule and folding the MHC heavy chain molecule in the presence of a peptide and a light chain polypeptide (e.g., a β2M light chain polypeptide). For example, an MHC heavy chain molecule and a β2M light chain polypeptide can be produced in E. coli, gel purified, diluted in the presence of peptide to form an MHC/peptide complex. See, e.g., Garboczi et al. (Proc. Nat. Acad. Sci., 89:3429-3433 (1992)), and Sylvester-Hvid et al. (Tissue Antigens. 59(4):251 (2002)). In some cases, soluble MHC heavy chain and β2M light chain can be produced and secreted in soluble form from mammalian cells. See, e.g., U.S. Patent Application No. 20060035338, and U.S. Patent Application No. 20060134744.

MHC/peptide complexes can be attached to, for example, a streptavidin molecule or a polymer backbone (e.g., polyacrylic amide, pullulan, or dextran). For example, biotinylated MHC/peptide complexes can be attached to a polyacrylic amide molecule to which streptavidin molecules have been attached. In other cases, an MHC/peptide multimer can comprise a dextran backbone (i.e., a dextramer) (Batard et al., J. Immunol. Meth. 310(1-2):136-148 (2006)). An MHC/peptide complex can comprise an appropriate label, such as a fluorescent label, a chromatophore, an antigenic label, or an affinity tag (e.g., a biotin tag, or a magnetic tag).

An MHC/peptide complex can be further incorporated into an MHC/peptide multimer comprising two or more (e.g., 2, 3, 4, 5, 6, 7, or more) different MHC/peptide complexes. See, for example, WO 02072631, which describes methods for making multimers. The number and type of MHC/peptide complexes, as well as the label used, can be adjusted based on the intended use of the MHC/peptide multimer. For example, an MHC/peptide multimer to be used on samples from a varied population of patients can comprise a greater number of different MHC/peptide complexes than an MHC/peptide multimer to be used on a less varied population of patients.

An MHC/peptide complex can be used to enumerate the numbers of antigen-specific T cells (e.g., CD8+ T cells, or CD 4+ T cells) in a biological sample (e.g., a blood sample, a lymph sample, a bone marrow sample, a spleen biopsy, or a sample from a splenectomy) from a subject. MHC/peptide complexes can be used in any suitable assay. For example, an MHC/peptide complex can be labeled with a fluorescent label and used in a flow cytometry-based assay. In such an assay, T cells from a biological sample can be contacted with a labeled MHC/peptide complex and T cells that are bound to an MHC/peptide complex can be detected by flow cytometry. See, e.g., Storm et al. (J. Virol. 80(3):1222-1230 (2006)), and Appay and Rowland-Jones (J. Immunol. Methods. 268(1):9-19 (2002)).

Labeled antibodies can be used to evaluate whether T cells bound to an MHC/peptide complex express a T cell activation marker (e.g., a cytokine) or a cytotoxic (e.g., cytolytic) function marker. For example, if CD8+ T cells are used, antibodies such as anti-interferon (IFN)-gamma, anti-tumor necrosis factor (TNF)-alpha, anti-interleukin (IL)-2, anti-macrophage inflammatory protein (MIP)-1 alpha, or anti-MIP-1 beta can be used to detect activation markers such as IFN-gamma, TNF-alpha, IL-2, MIP-1 alpha, or MIP-1 beta, respectively, while antibodies such as anti-CD107a and anti-CD107b can be used to detect cytotoxic function markers such as CD107a (lysosomal-associated membrane protein (LAMP)-1) and CD107b (LAMP-2), respectively. See, e.g., Appay and Rowland-Jones (J. Immunol. Methods. 268(1):9-19 (2002)), Kallas et al (J. Infect. Dis. 179:1124-1131 (1999)), Betts et al (J. Immunol. Methods. 281(1-2):65-78 (2003)), and Hamann et al (J. Exp. Med. 186(9):1407-1418 (1997)).

One or more MHC/peptide complexes can also be used in an ELISA-based assay, such as an ELISPOT assay (Czerkinsky et al. (J Immunol Methods. 65(1-2):109-21 (1983)). See, e.g., Tissot et al. (J Immunol Methods. 236(1-2):147-65 (2000)). In some embodiments, one or more MHC/peptide complexes can be attached to a surface. For example, T cells can be contacted with one or more MHC/peptide complexes that are attached to a surface such as glass. T cell activation markers (e.g., IFN-gamma, TNF-alpha, IL-2, MIP-1 alpha, MIP-1 beta, CD107a, and CD107b) expressed by T cells that are bound to an MHC/peptide complex can be detected using a labeled antibody that specifically binds to a T cell activation marker (e.g., IFN-gamma, TNF-alpha, or IL-2, MIP-1 alpha, MIP-1 beta, CD107a, or CD107b). In other embodiments, one or more MHC/peptide complexes can remain soluble. For example, T cells can be contacted with one or more MHC/peptide complexes and then contacted with an antibody that specifically binds to a T cell activation marker or a cytotoxic function marker and that is attached to a surface. Activated T cells can be enumerated by detecting the expression of a T cell activation marker using a standard ELISPOT assay.

The peptides and compositions described herein can be used to determine or evaluate BKV-specific CD8+ T cell immune competence in a subject. BKV-specific CD8+ T cell immune competence (e.g., immunocompetence vs. immunodeficiency) can be evaluated based on the level of a BKV-specific CD8+ T cell parameter. A BKV-specific CD8+ T cell parameter can include 1) the percentage of CD8+ T cells having receptors that bind an MHC/(BKV MHC Class I epitope peptide) complex, 2) the amount of IFN-gamma, TNF-alpha, IL-2, MIP-1 alpha, or MIP-1 beta produced by CD8+ T cells that are exposed to an MHC/(BKV MHC Class I epitope peptide) complex, 3) the cytolytic activity of CD8+ T cells that are exposed to an MHC/(BKV MHC Class I epitope peptide) complex, or a combination thereof. The percentage of CD8+ T cells having receptors that bind an MHC/(BKV MHC Class I epitope peptide) complex can be determined using methods known in the art, including, those provided herein. The amount of IFN-gamma, TNF-alpha, IL-2, MIP-1 alpha, or MIP-1 beta produced by CD8+ T cells that are exposed to an MHC/(BKV MHC Class I epitope peptide) complex can be determined using known methods such as ELISA-based assays or intracellular staining techniques in combination with flow cytometry. The potential cytolytic activity (based on degranulation) of CD8+ T cells that are exposed to an MHC/(BKV MHC Class I epitope peptide) complex can be determined using, e.g., extracellular staining for CD107a and CD107b (Betts et al. (J. Immunol. Methods. 281(1-2):65-78 (2003)) or by measuring the ability of activated CD8+ T cells to lyse labeled target cells in vitro (Ostler et al. (J Immunol Methods. 257(1-2): 155-61 (2001)).

T cells can be obtained from any appropriate tissue samples (e.g., blood, a lymph node, or spleen) and purified using standard procedures. For example, T cells can be selected based on cell surface markers such as CD4, CD8, and CD3, using fluorescence activated cell sorting or magnetic cell sorting. See, e.g., T Cell Protocols: Development and Activation (Methods in Molecular Biology). Kearse (ed.) Humana Press. pp. 63-68 and 133-142 (2000).

The minimum parameter levels indicative of BKV immunocompetence can be determined by comparing the parameters of CD8+ T cells from a population of subjects that have BKV-associated nephropathy that are contacted with a BKV MHC Class I epitope/MHC complex to the parameters of control CD8+ T cells (i.e., CD8+ T cells from a population of control subjects that do not have BKV-associated nephropathy or CD8+ T cells from a population of control subjects that are not BK infected) that are contacted with a BKV MHC Class I epitope/MHC complex. A subject can be termed BKV immunocompetent if the BKV-specific CD8+ T cell parameters of that subject's CD8+ T cells is greater than, or equal to, the minimum parameters required for BKV immunocompetence. Conversely, a subject can be termed BKV immune incompetent if the a BKV-specific CD8+ T cell parameters of that subject's CD8+ T cells is less than the minimum parameters required for BKV immunocompetence.

Also provided herein are articles of manufacture. An article of manufacture provided herein can be a kit containing one or more of the provided peptides in packaging material. In some cases the peptides can be provided as MHC/peptide complexes. In some cases, the peptides can be provided as MHC/peptide multimers. MHC/peptide multimers can be labeled and can be provided in soluble form or they can be attached to a surface (e.g., the surface of an ELISA plate). Kits can further contain labeled antibodies such as anti-IFN-gamma, anti-TNF-alpha, anti-MIP-1 alpha, anti-MIP-1 beta, anti-IL-2, anti-CD107a, or anti-CD107b.

Also provided herein are methods to treat, prevent, or ameliorate one or more symptoms of BKV reactivation (e.g., BKV-associated nephropathy) in a subject (e.g., a transplant patient) that is BKV immune incompetent. In some cases, the methods provided herein can be used to treat or ameliorate the symptoms of BKV reactivation or BKV-associated nephropathy in a patient that is BKV immune incompetent. For example, a subject that is BKV immune incompetent can be prescribed or administered an appropriate dose (i.e., a lower dose) of an immunosuppressive drug (e.g., cyclosporin, azathioprine, corticosteroid, tacrolimus, rapamycin, or mycophenolate mofetil) for a transplant (e.g., a kidney transplant or a bone marrow transplant) than a subject that is BKV immunocompetent, to treat or ameliorate the symptoms of BKV-associated nephropathy. In some cases, a subject that is BKV immune incompetent can be administered or prescribed an appropriate dose of an antiviral drug (e.g., cidifovir) to treat or ameliorate the symptoms of BKV-associated nephropathy.

The invention will be further described in the following examples, which do not limit the scope of the invention described in the claims.

### EXAMPLES

### Example 1

### Identifying BKV_MHC Class I Epitopes

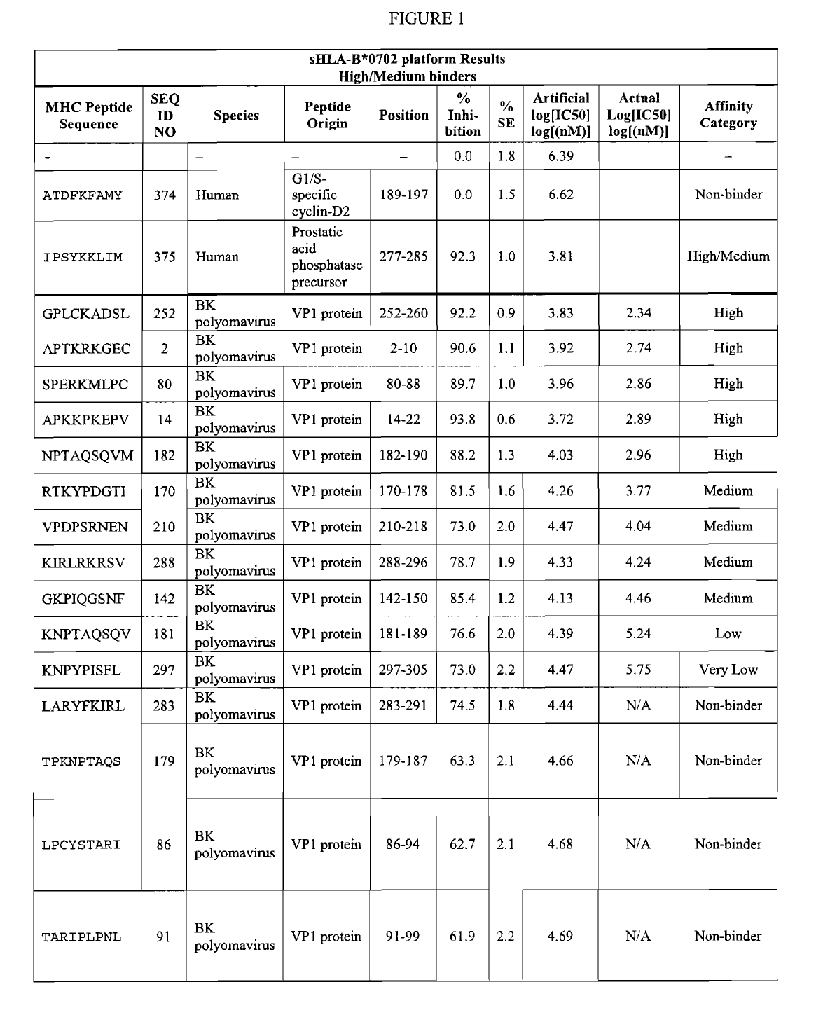

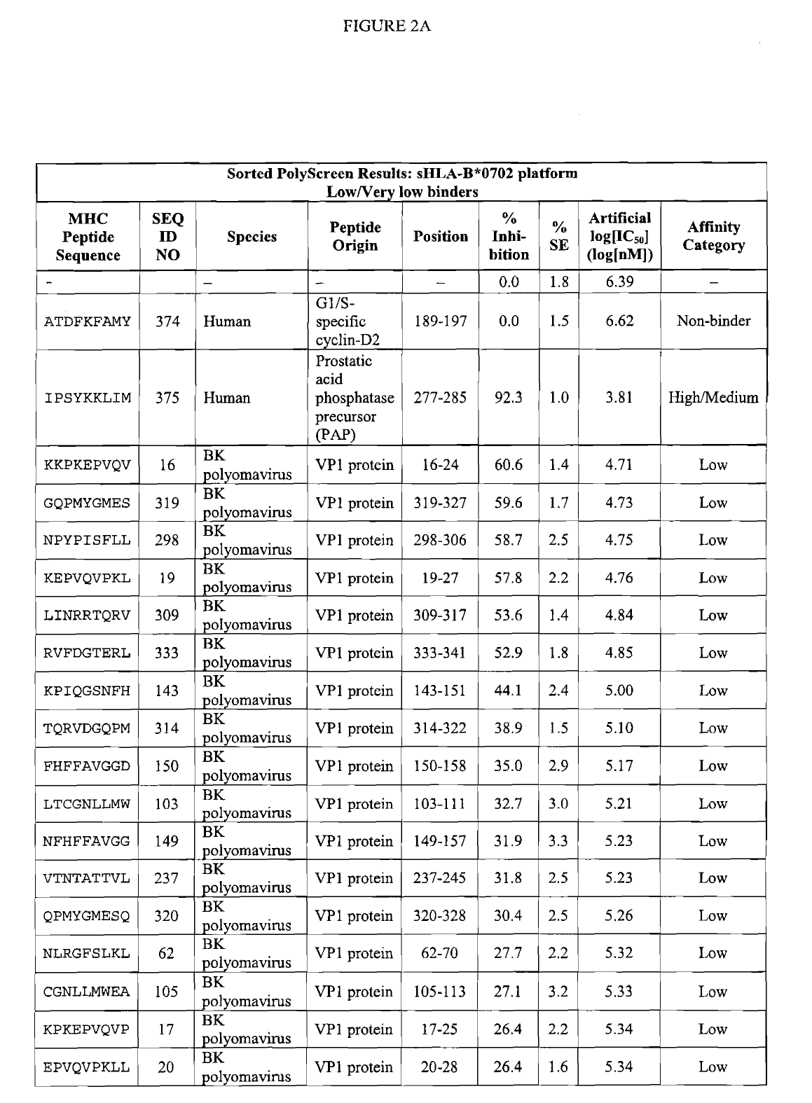

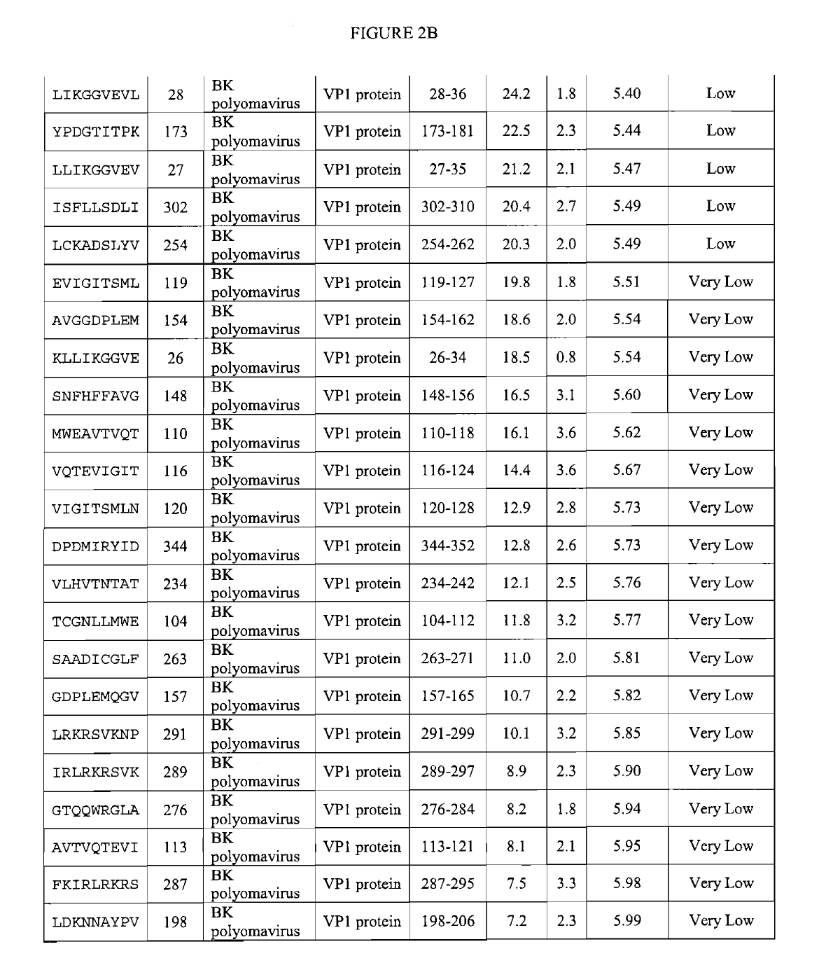

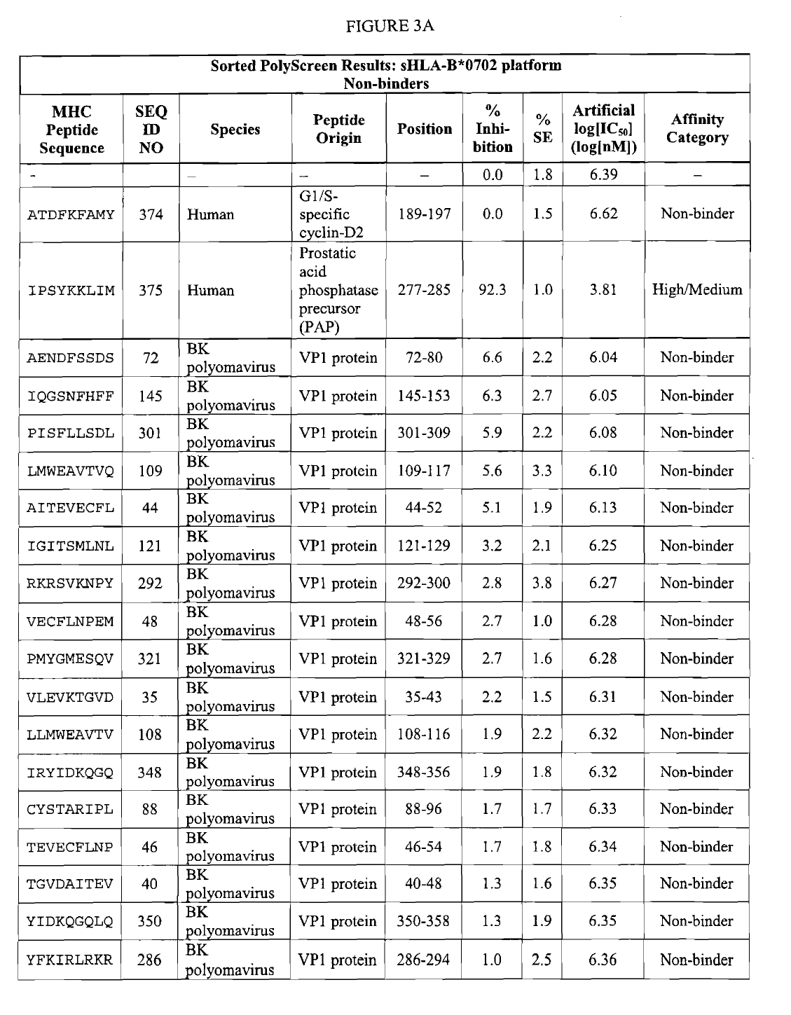

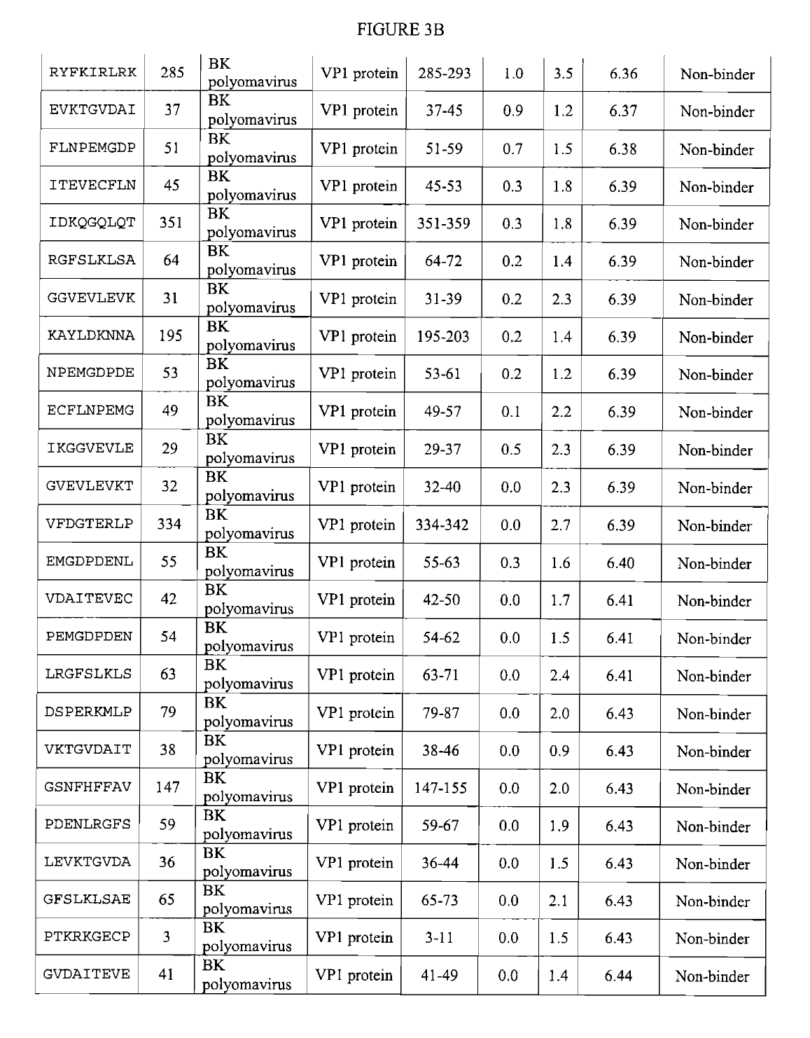

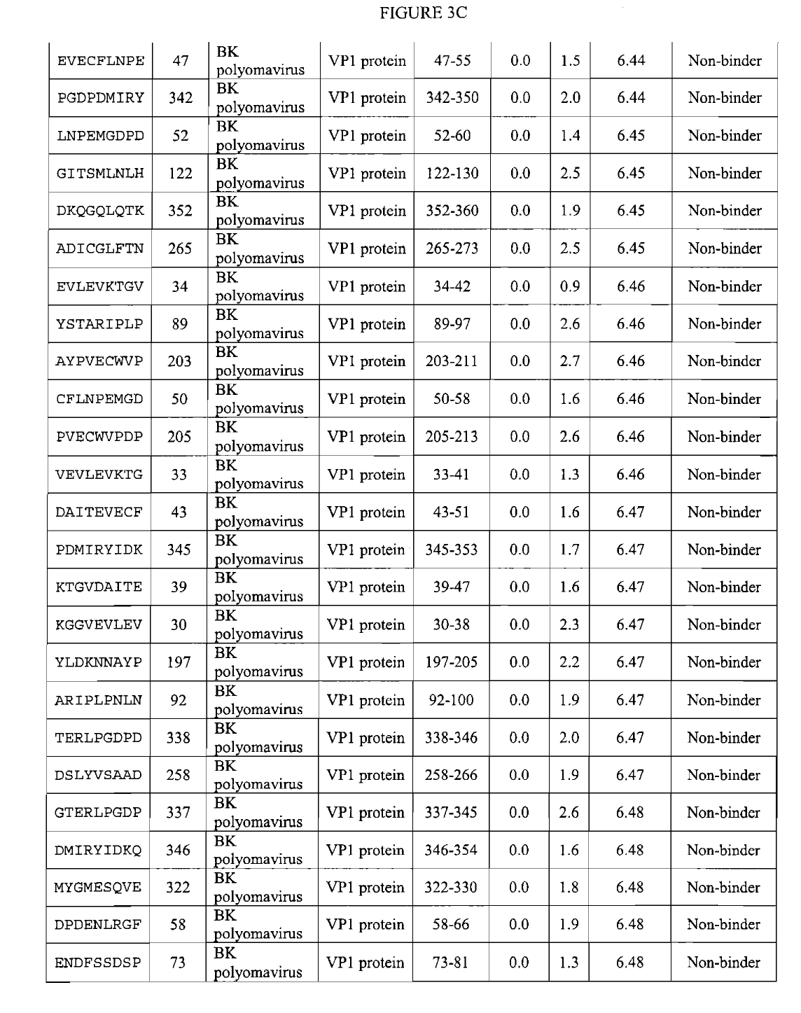

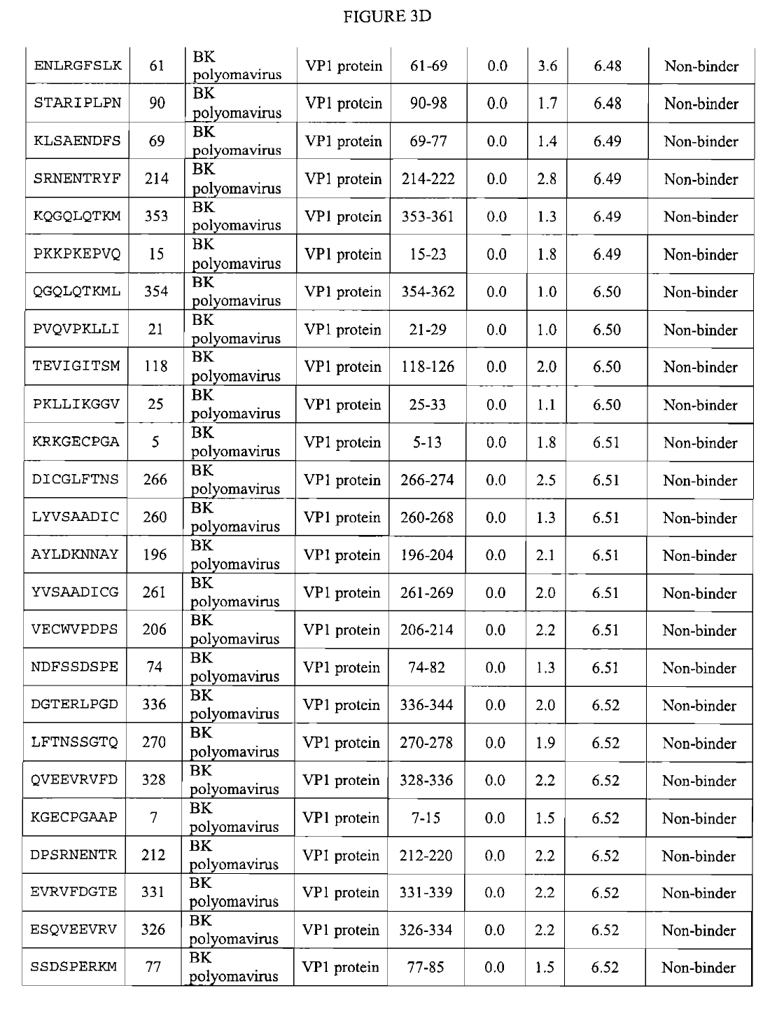

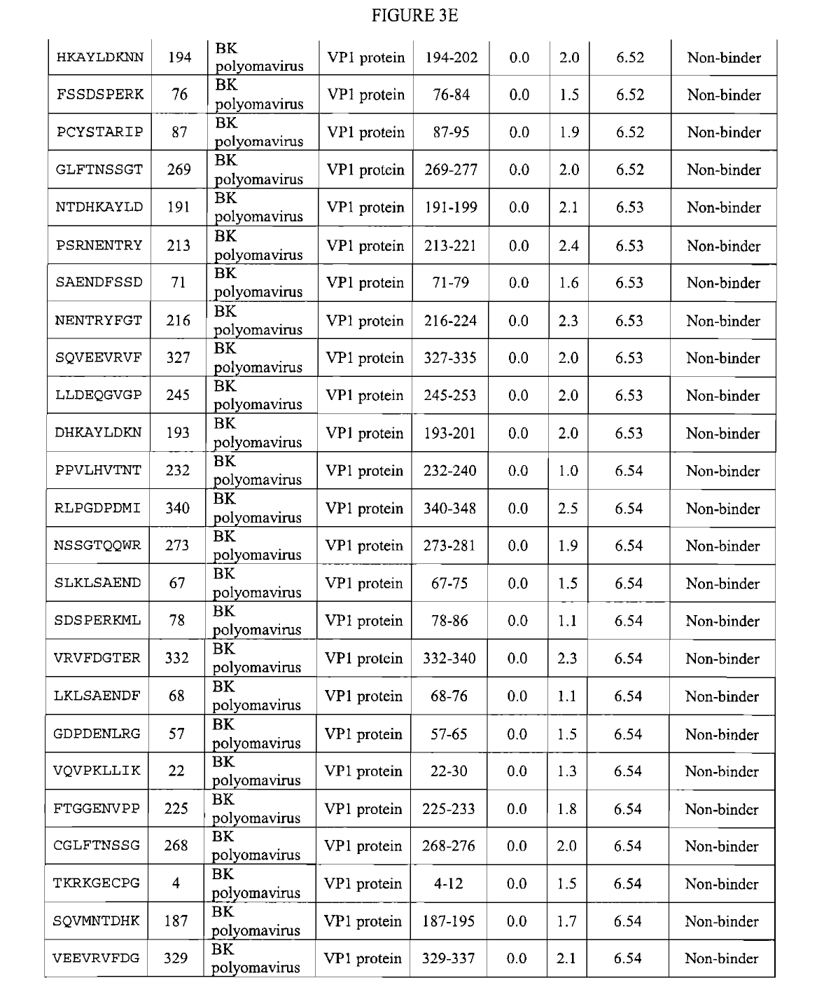

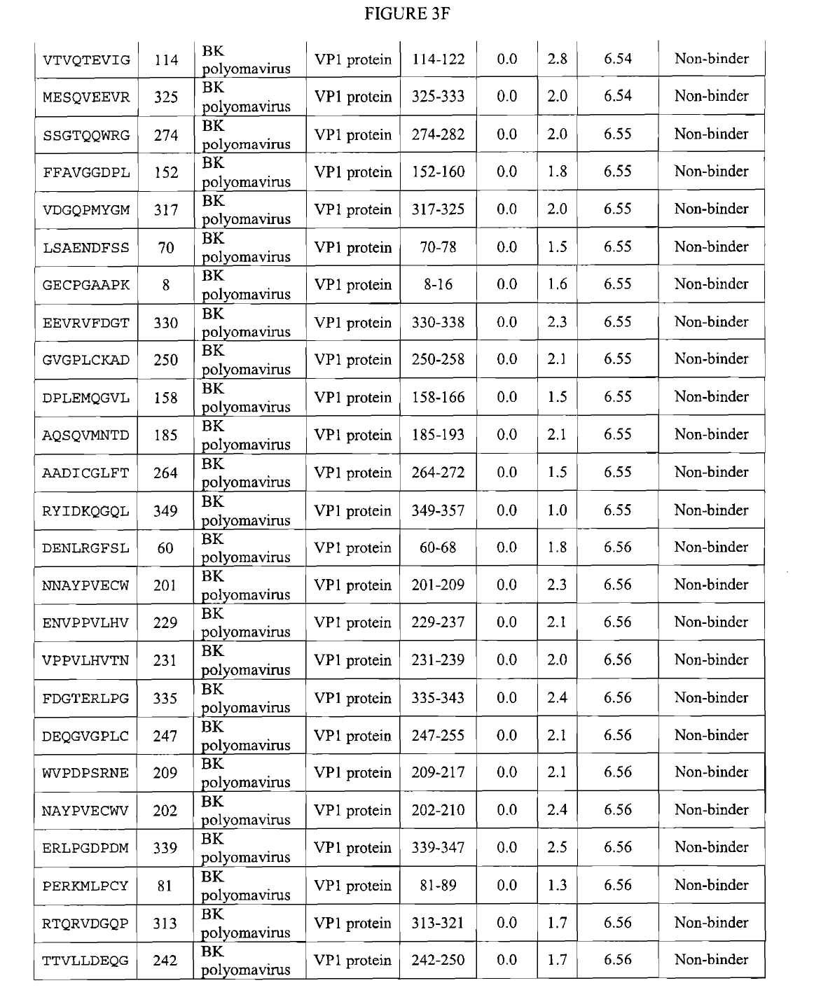

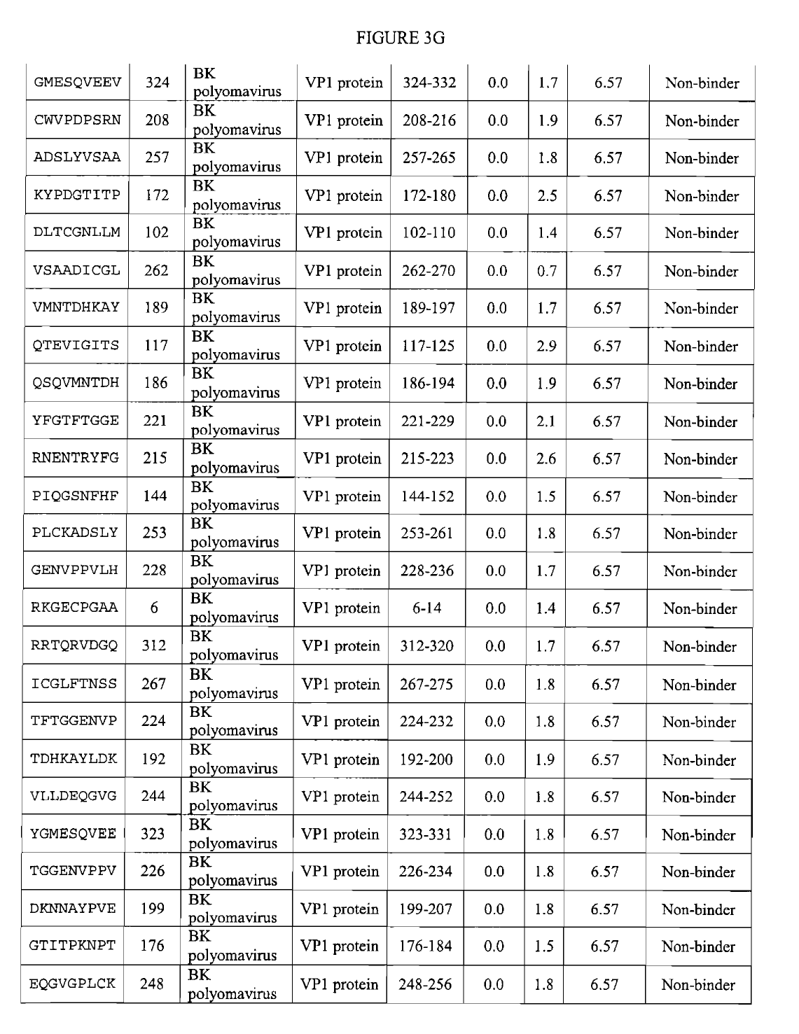

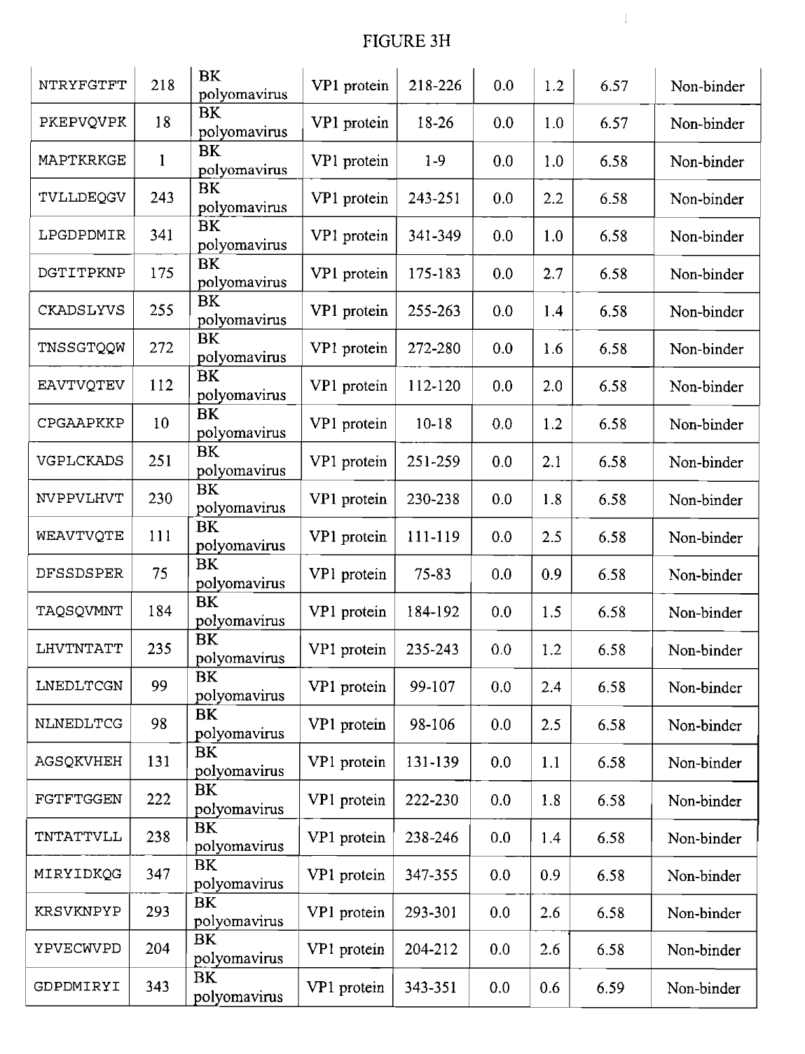

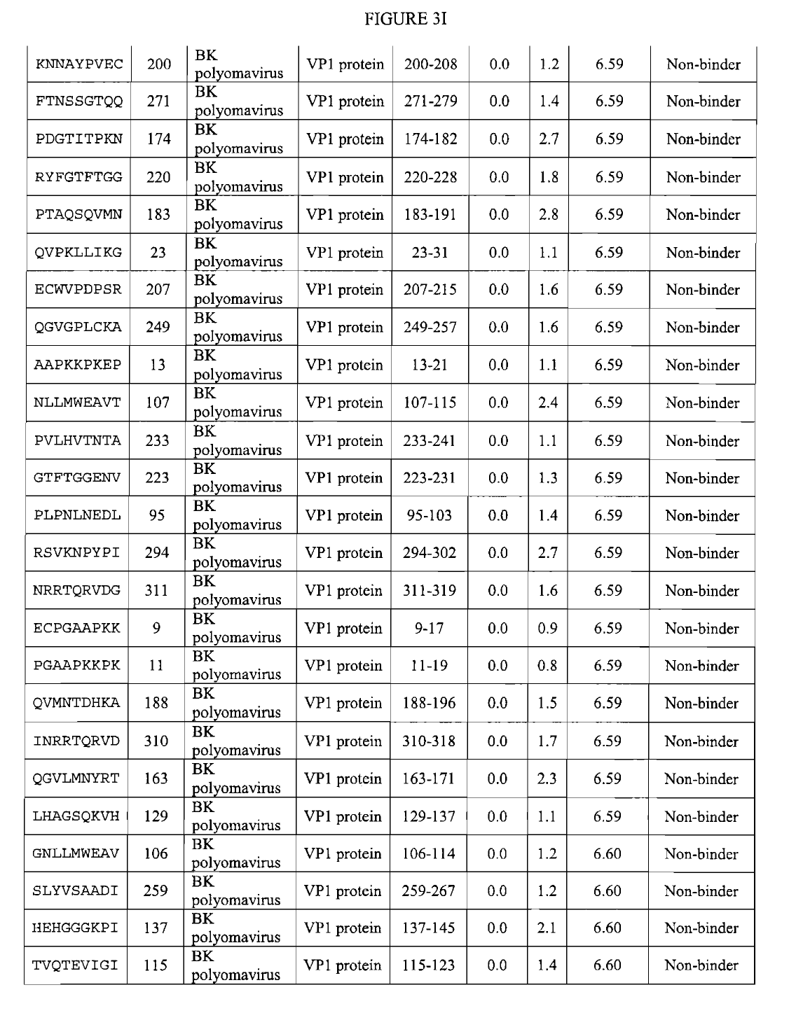

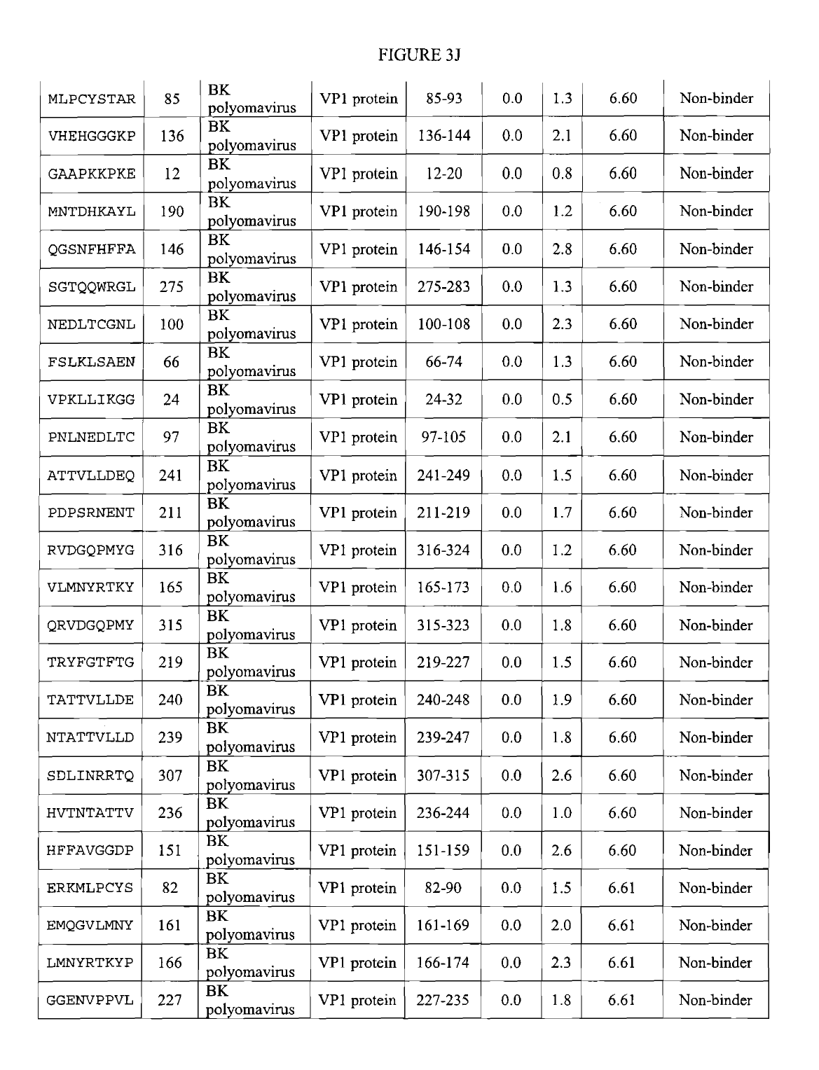

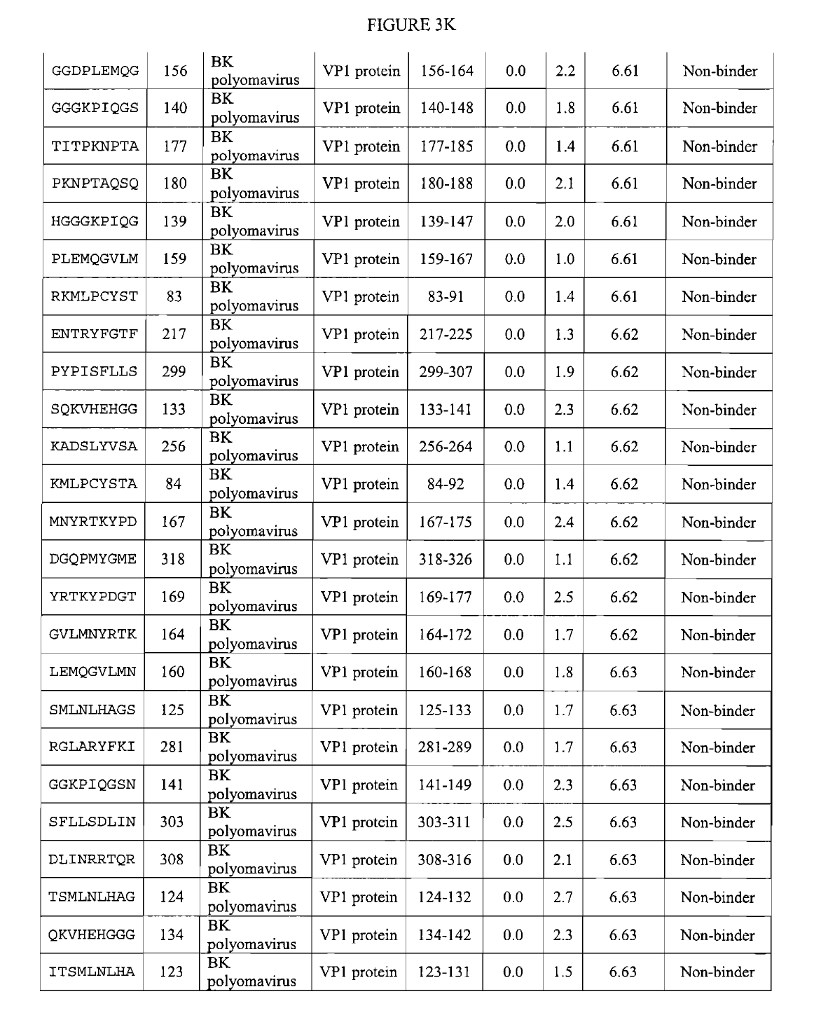

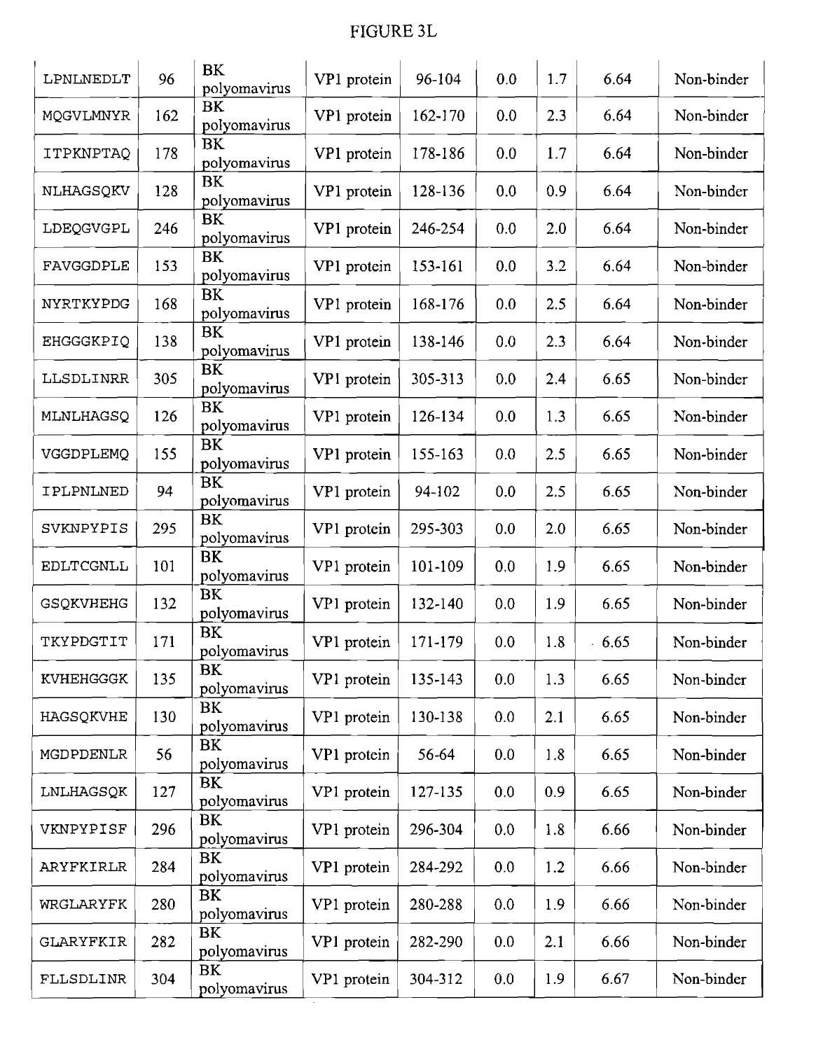

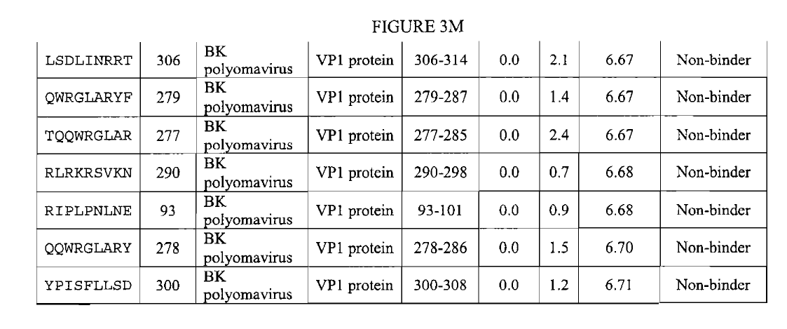

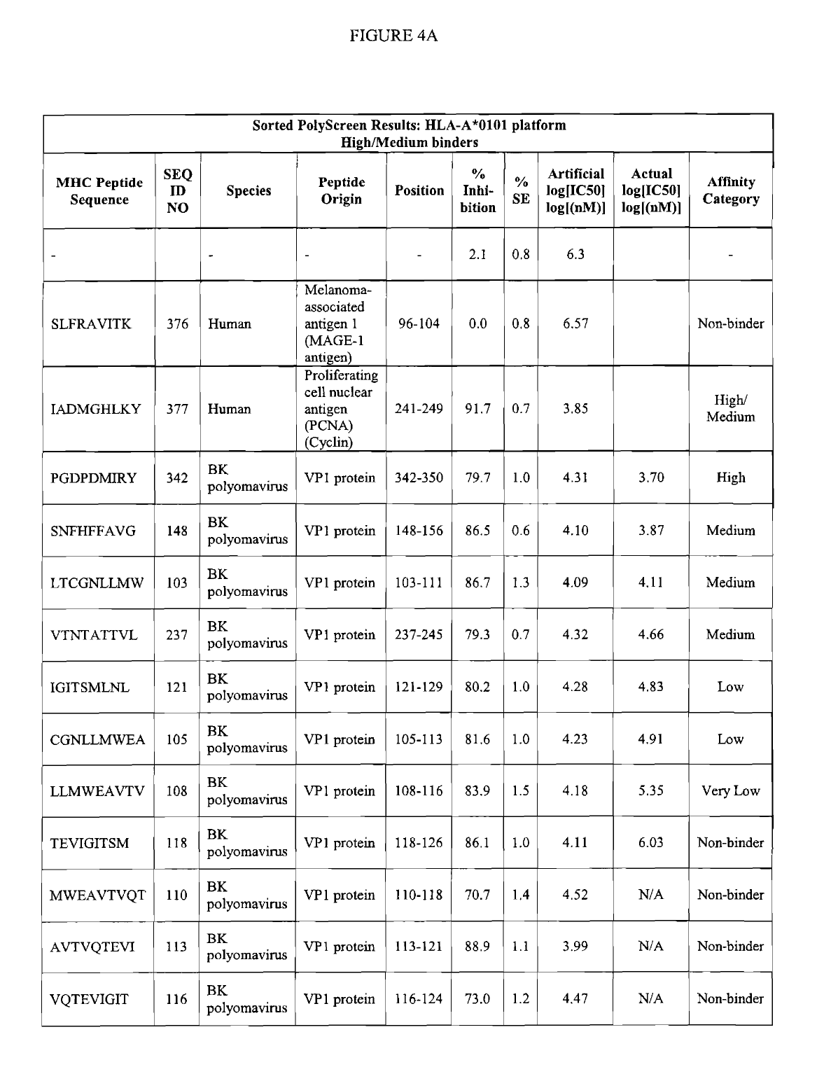

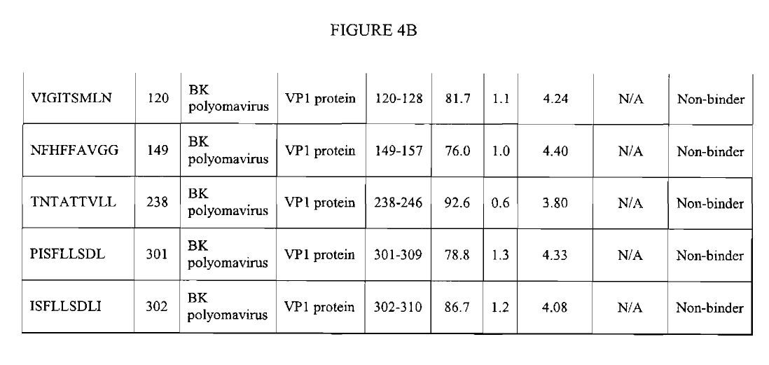

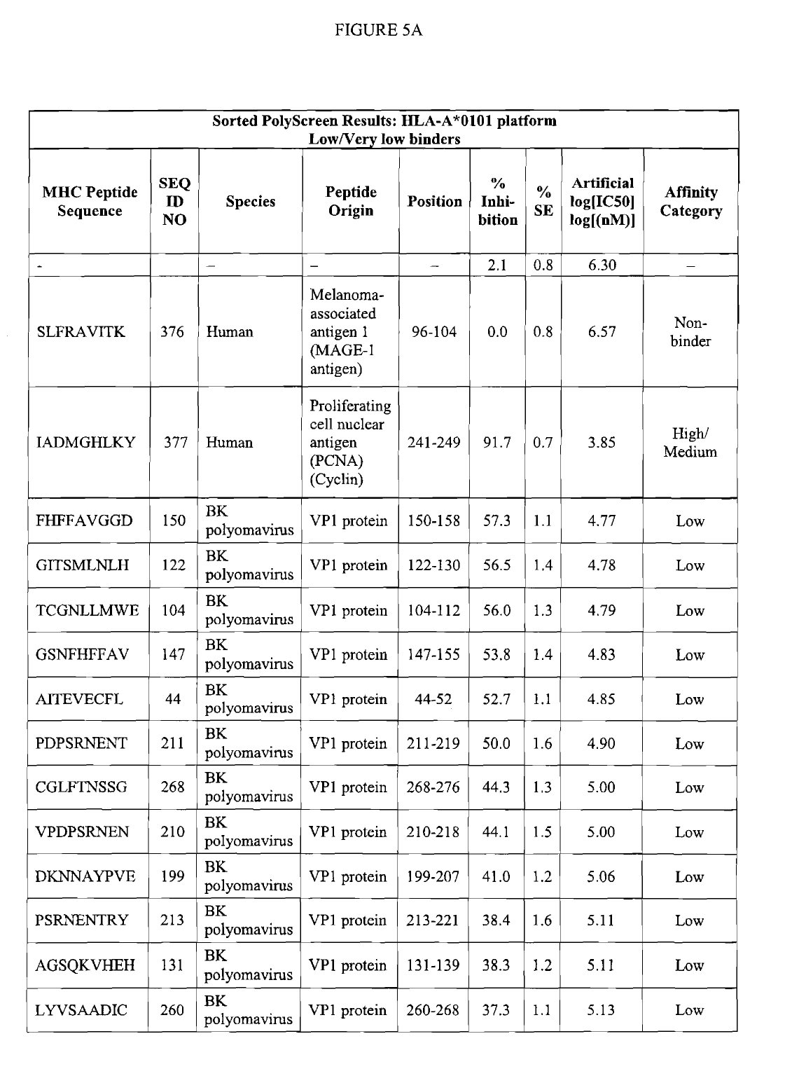

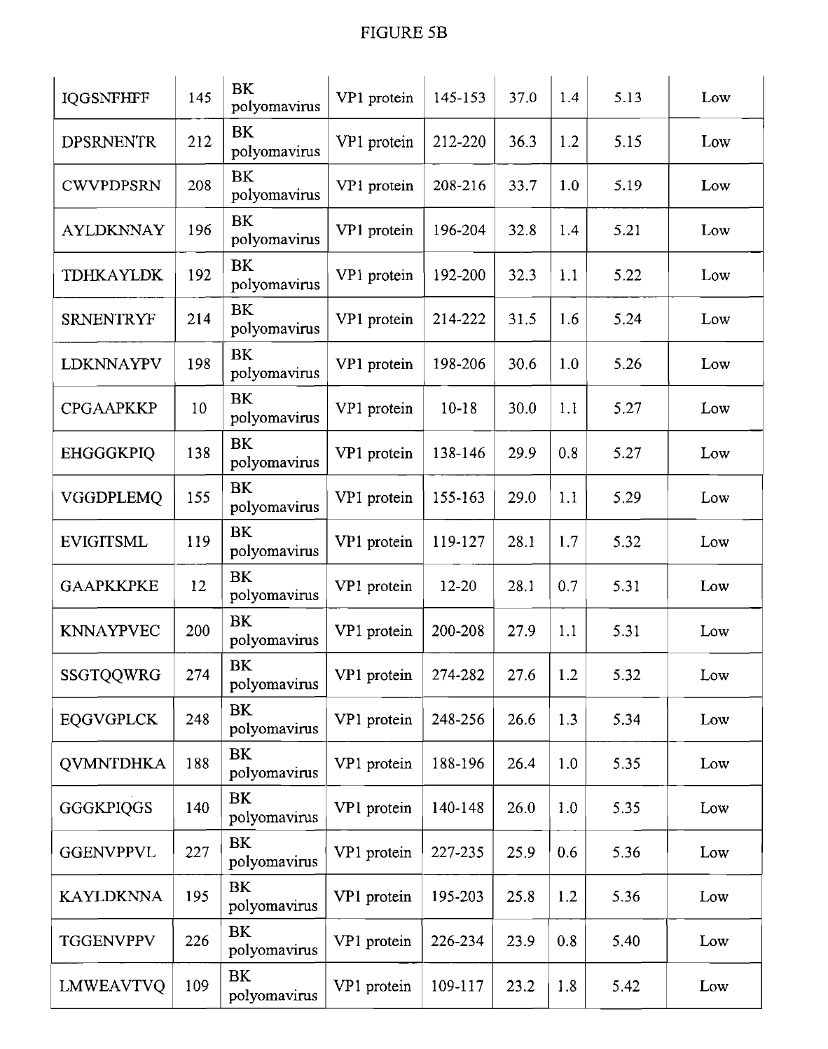

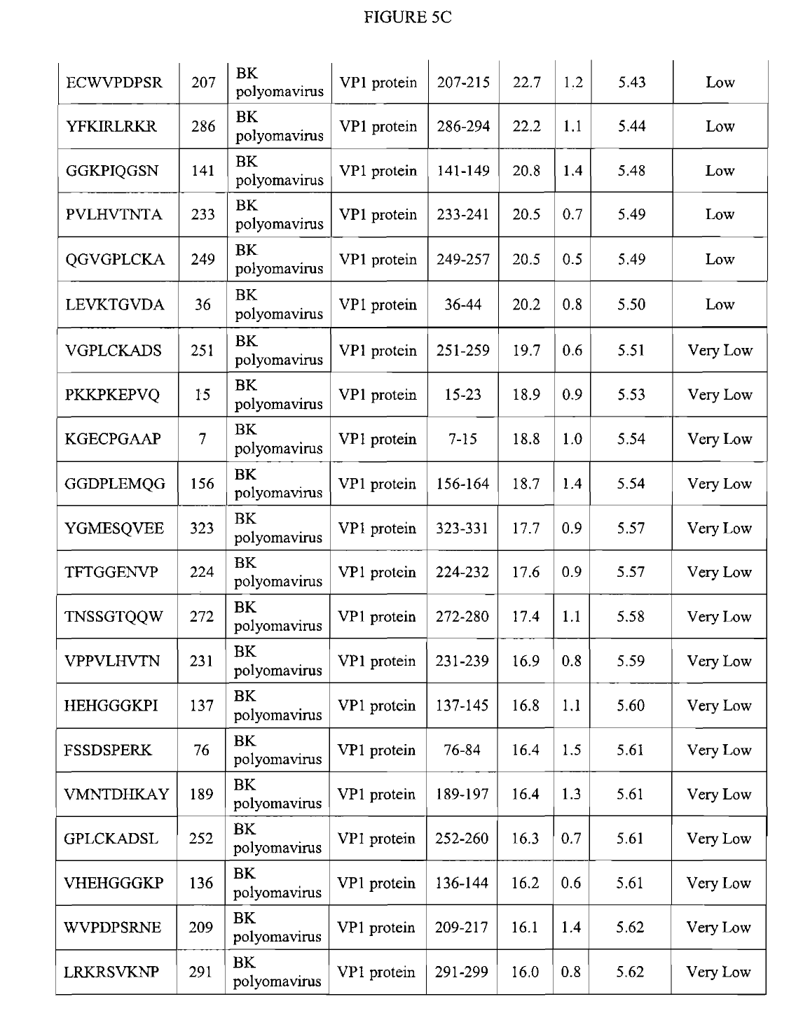

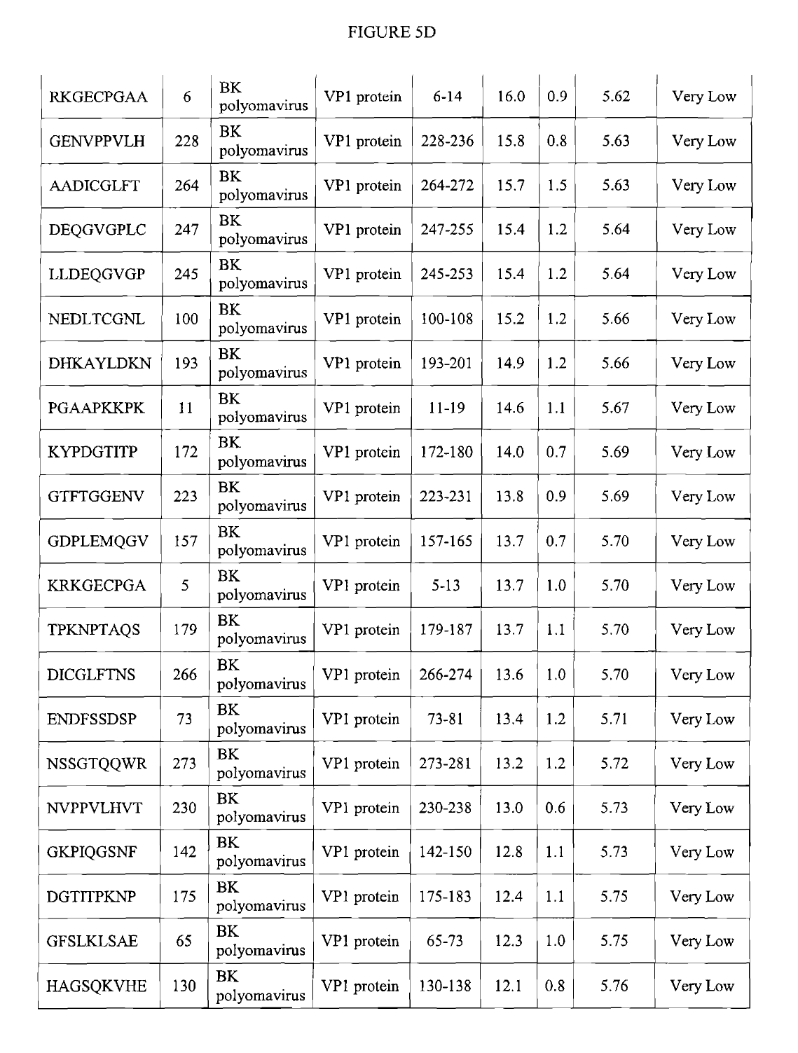

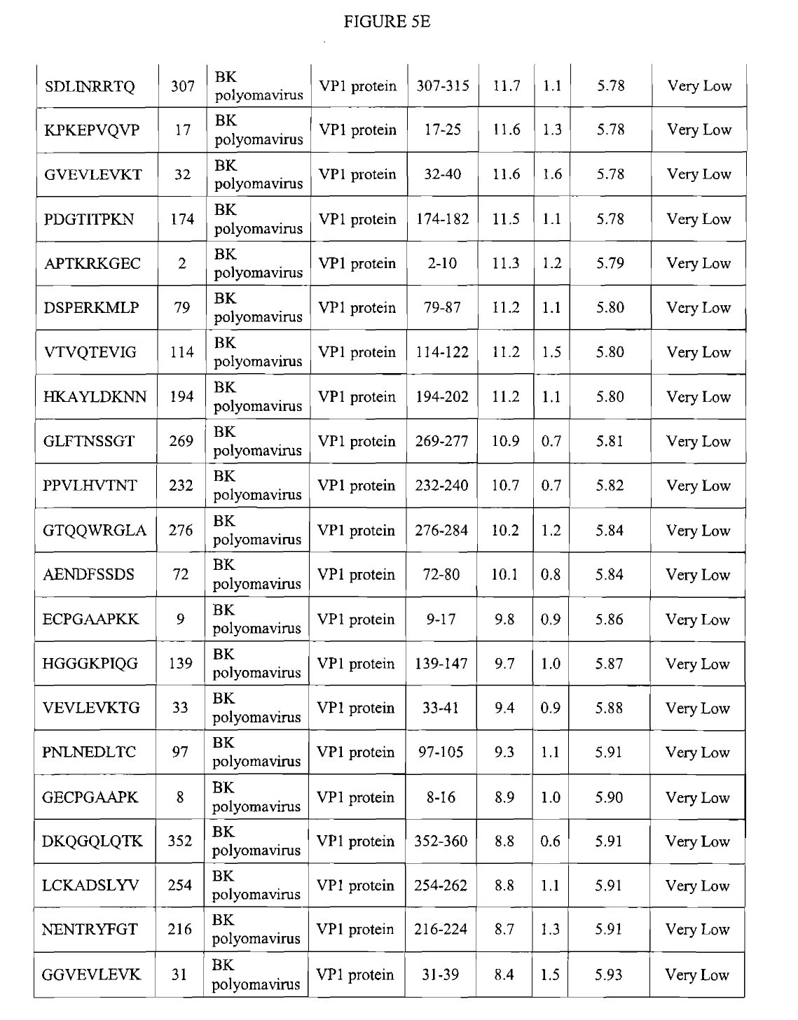

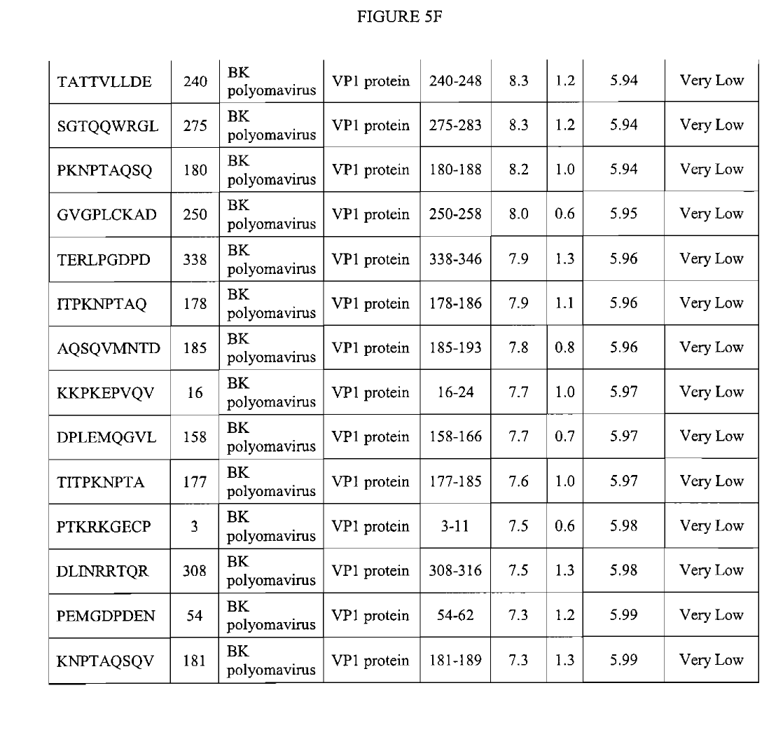

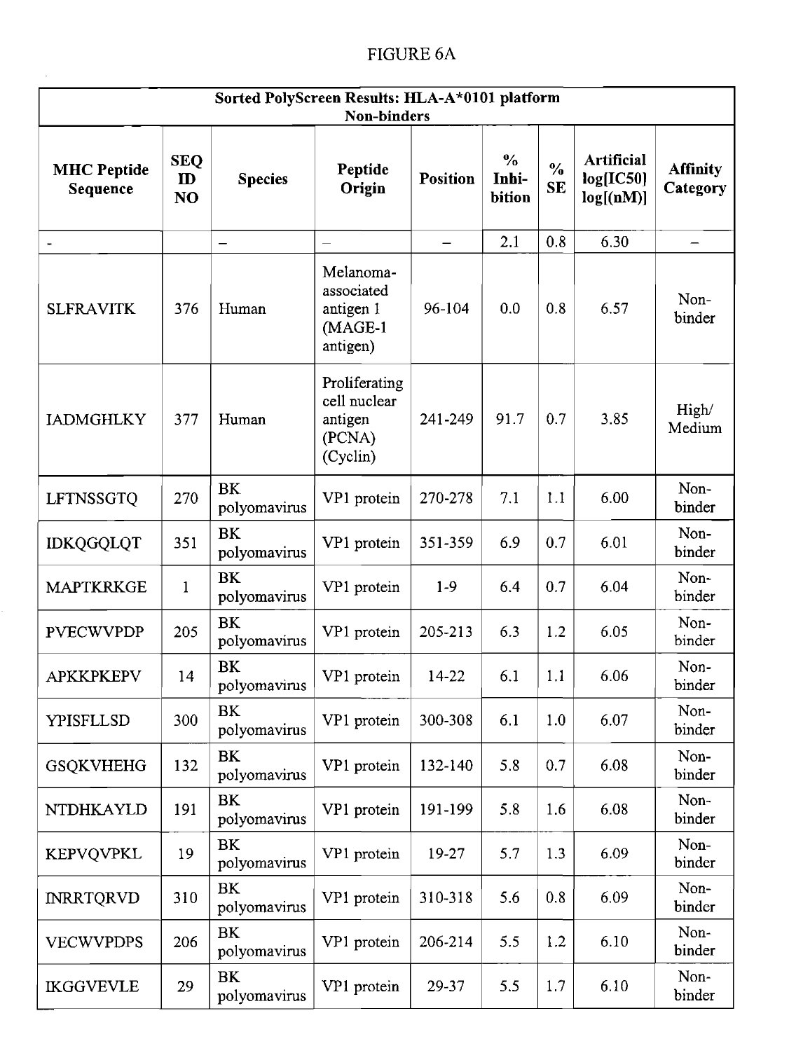

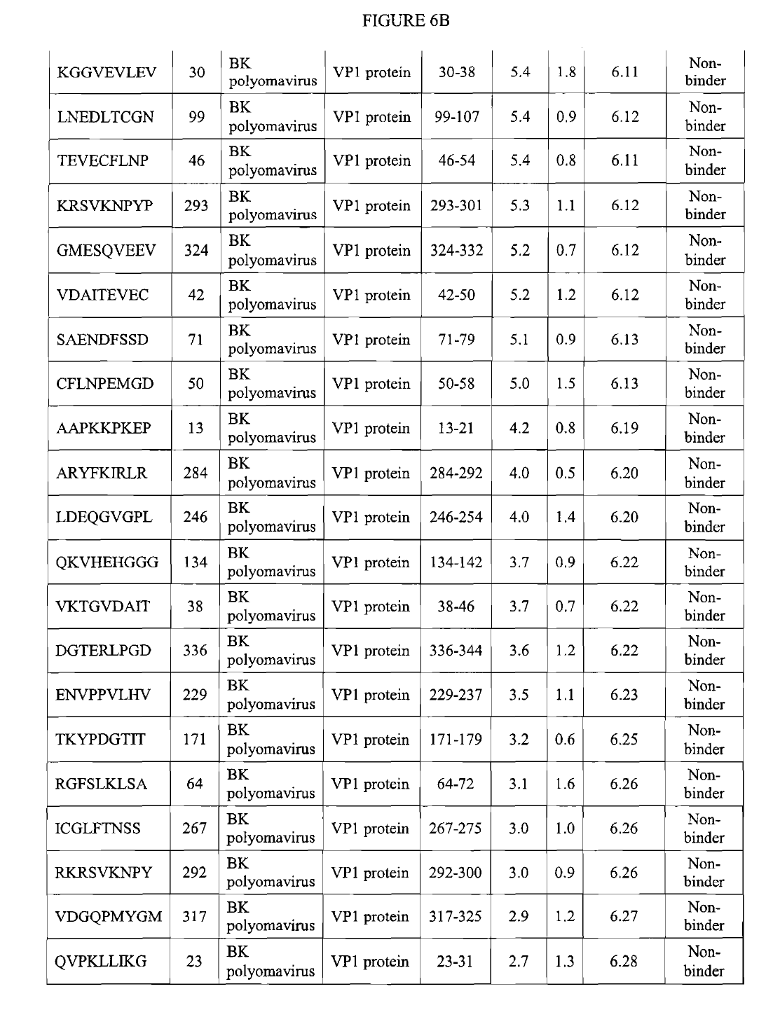

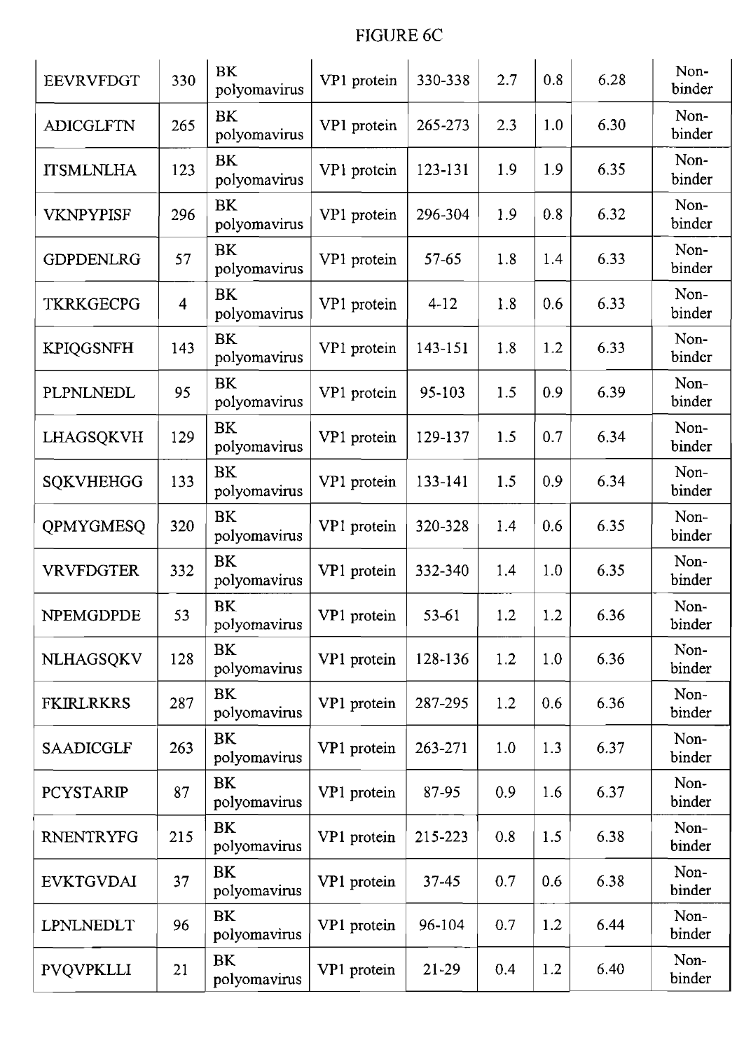

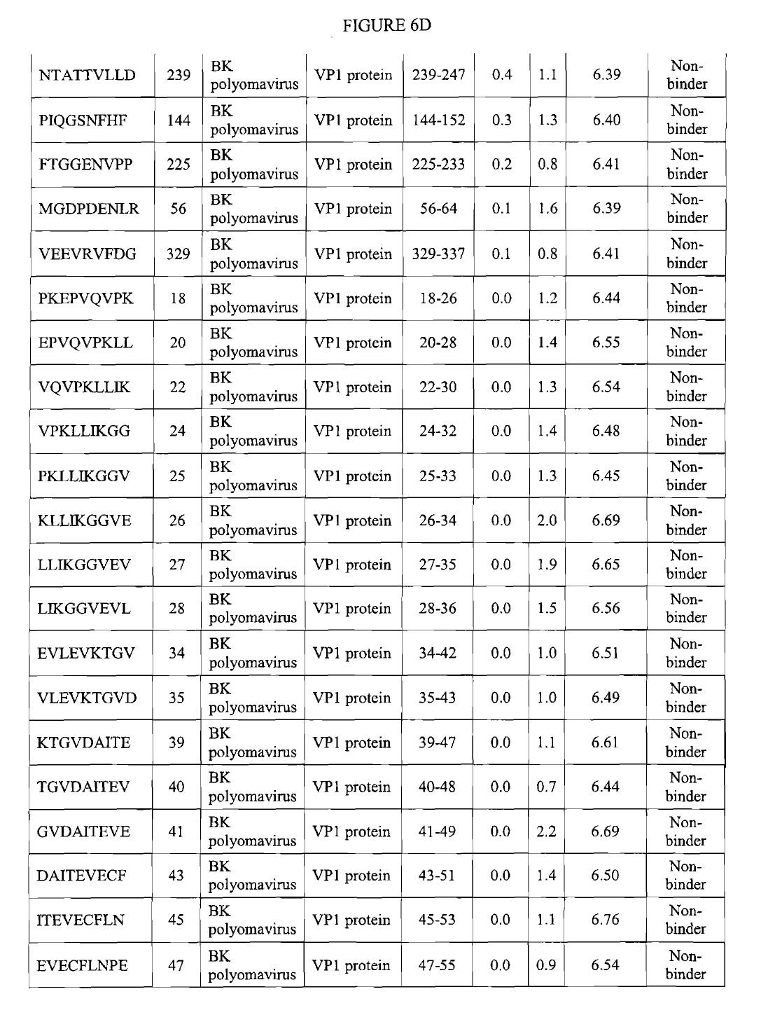

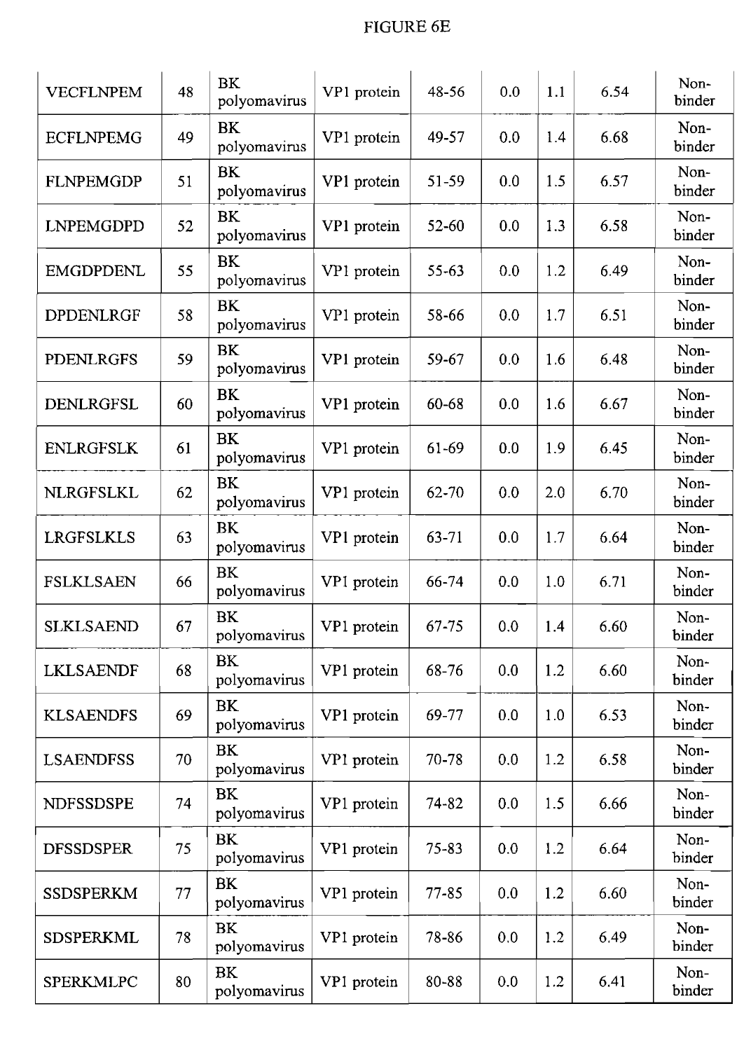

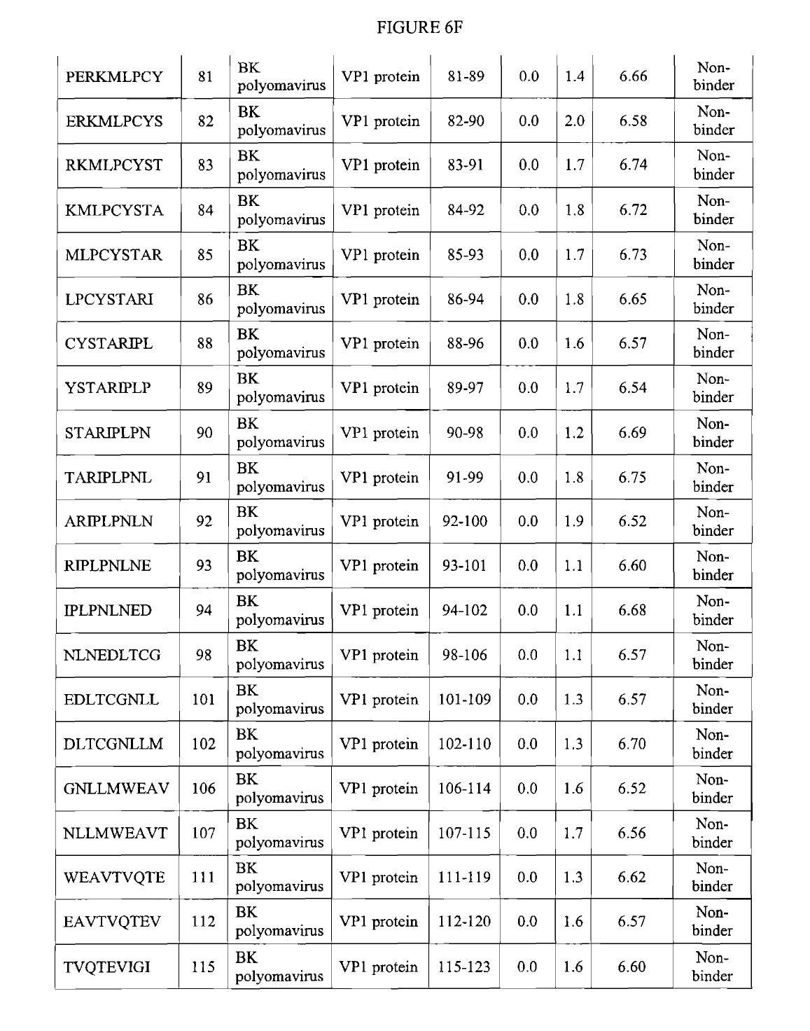

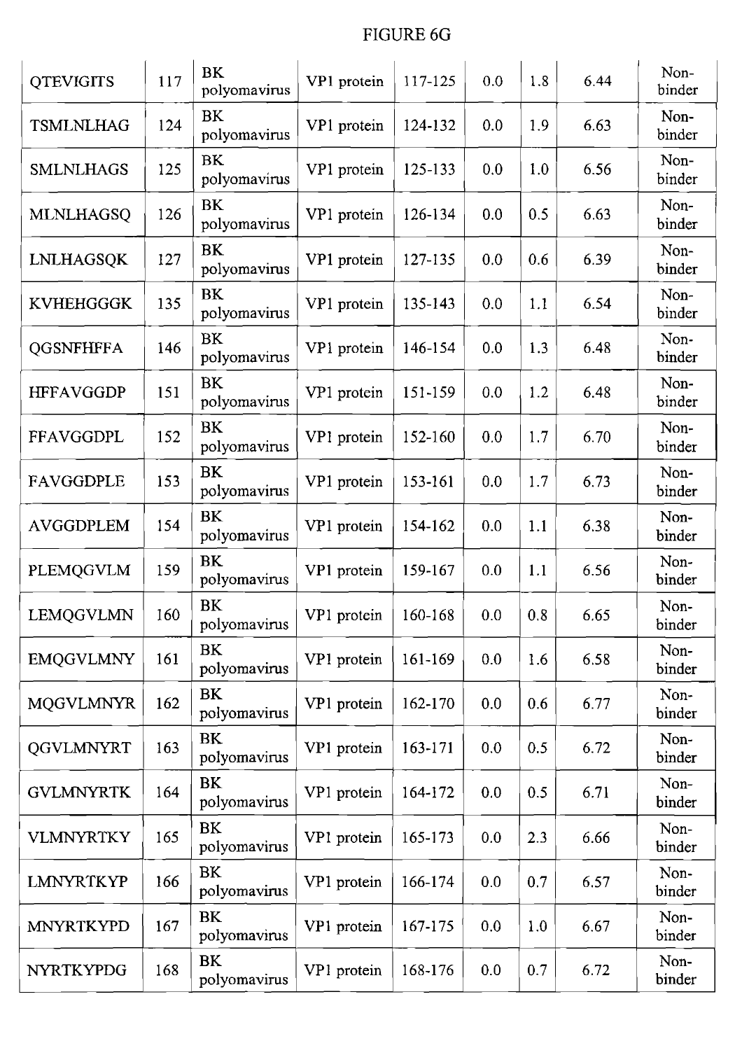

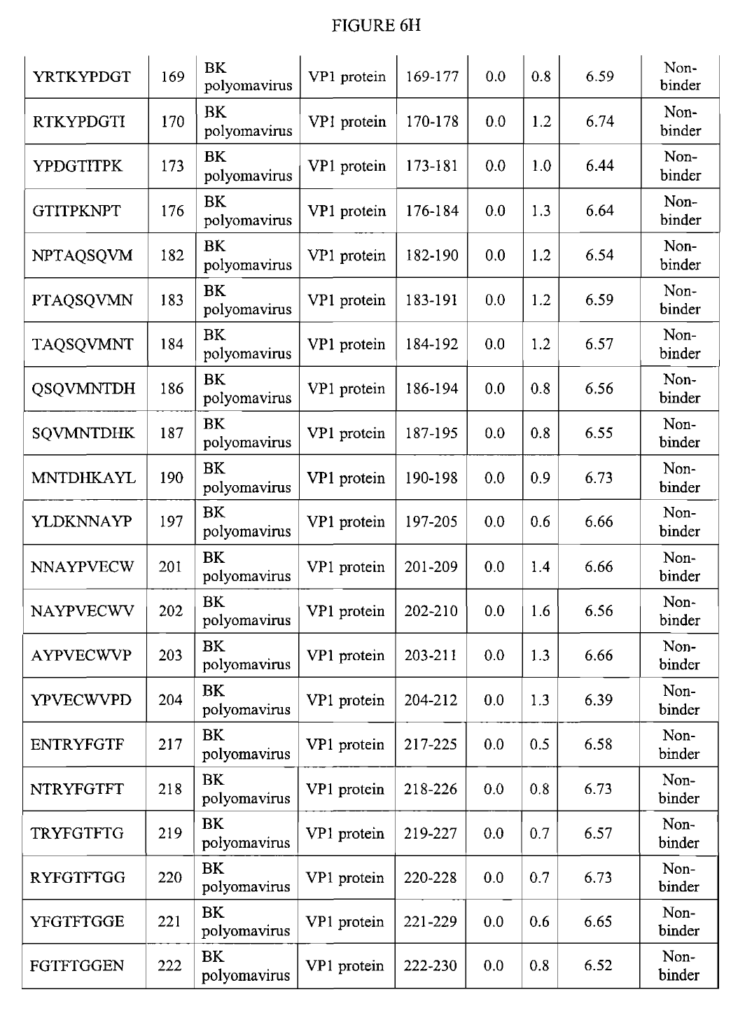

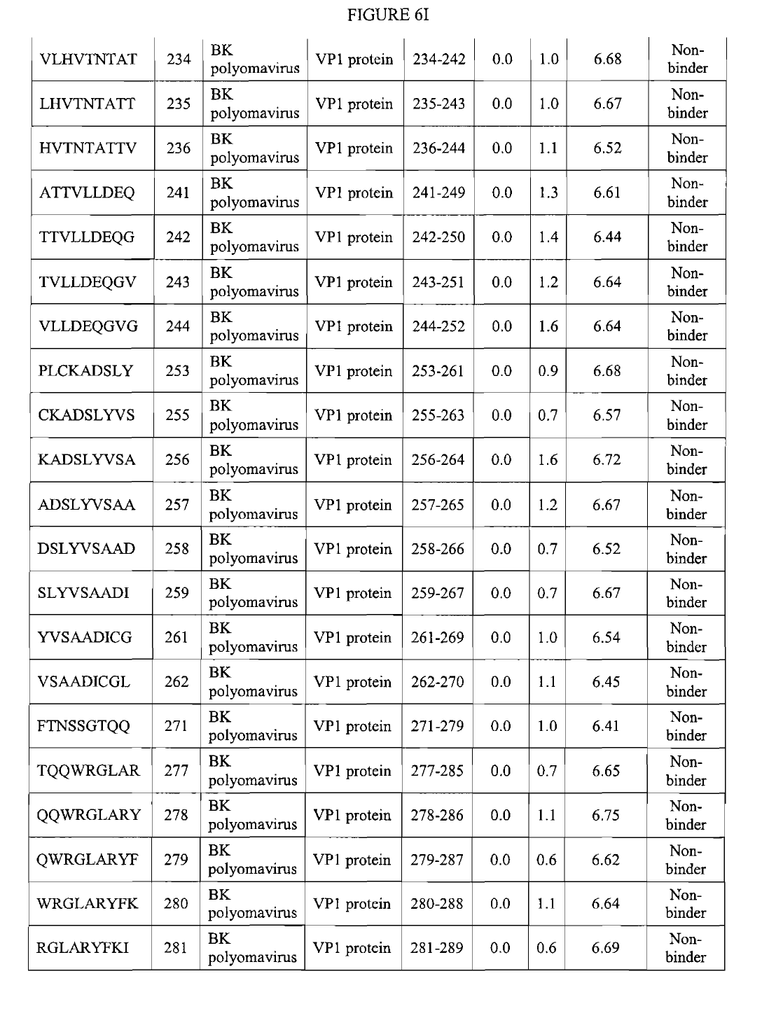

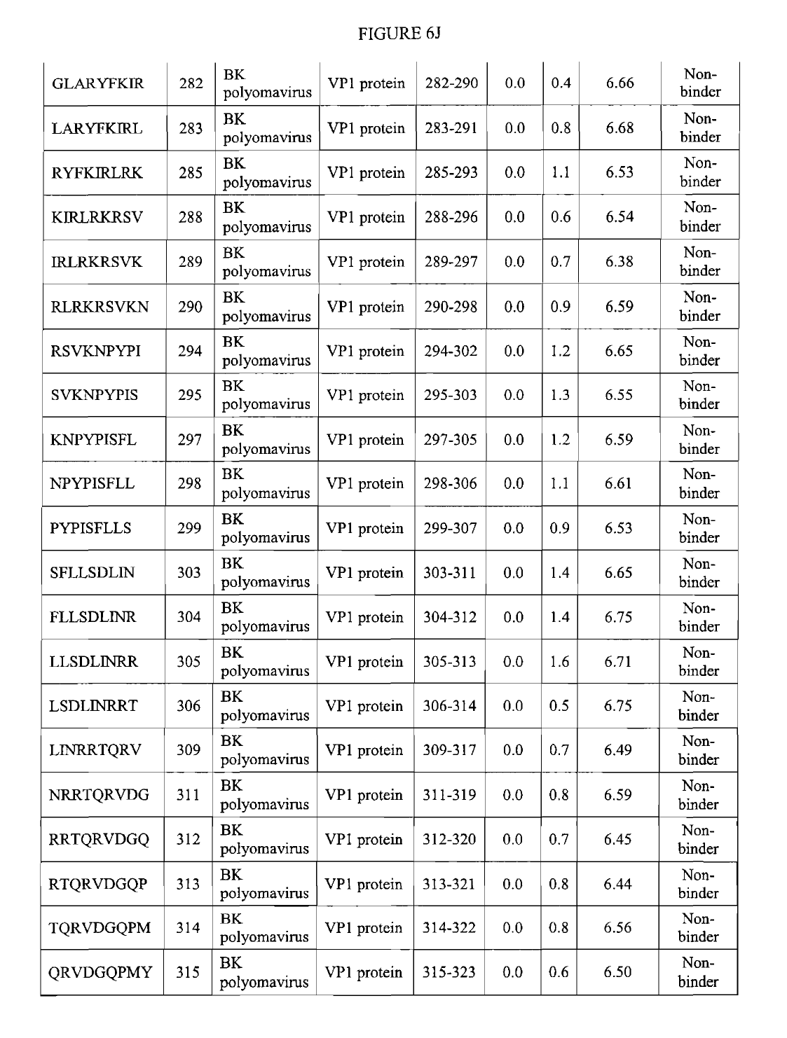

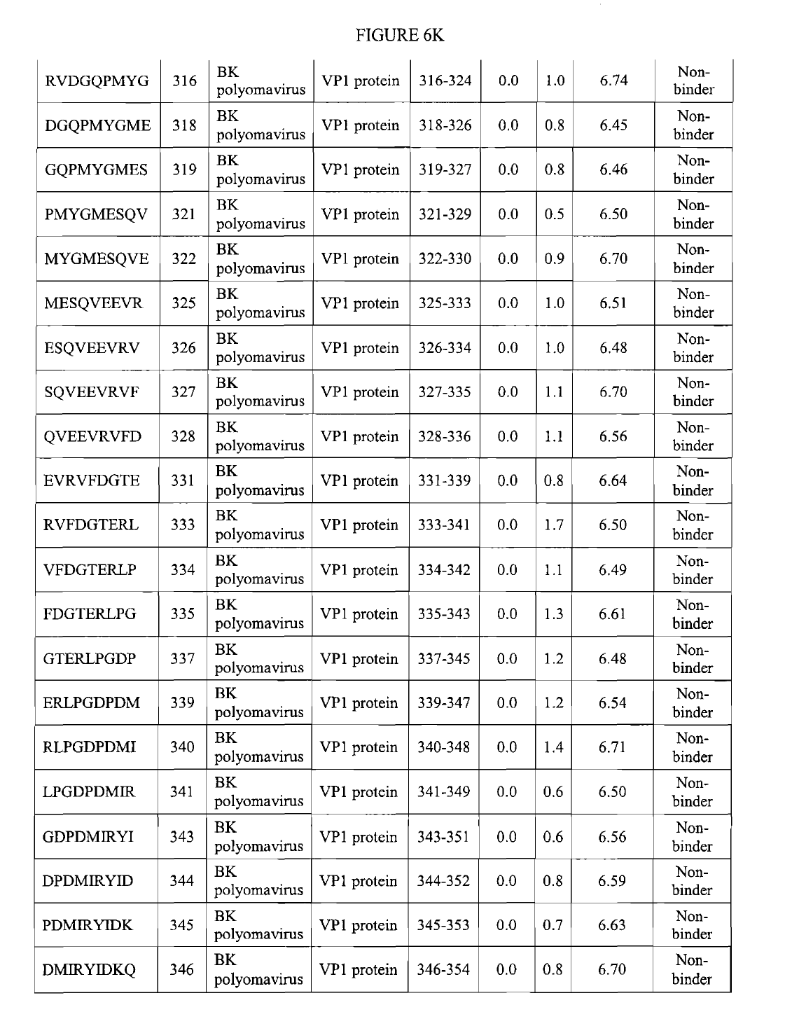

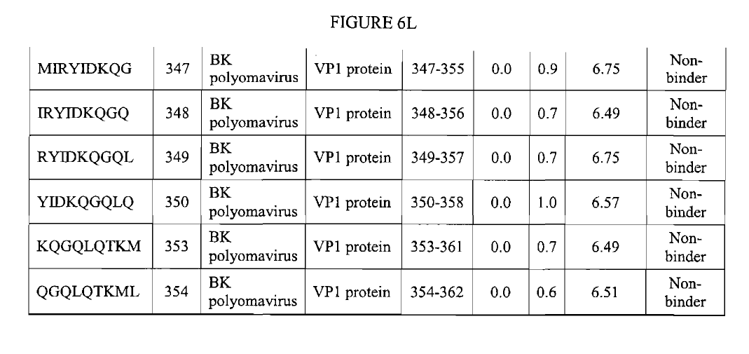

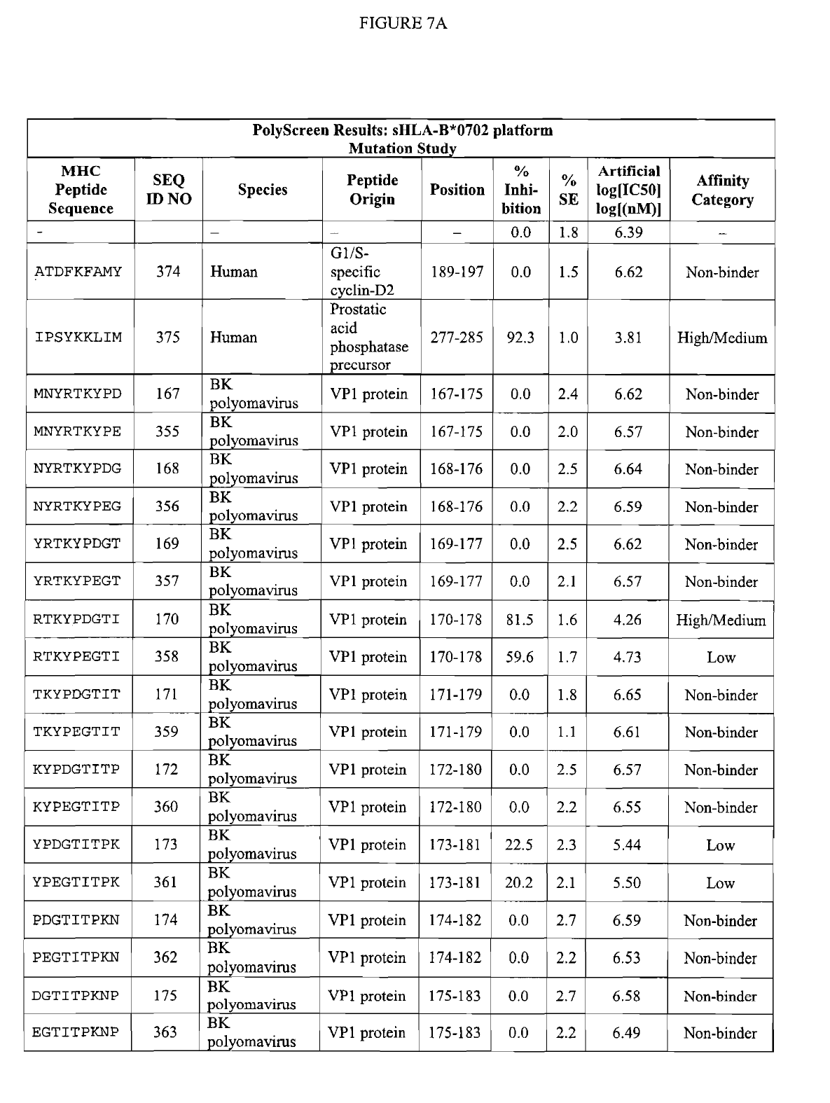

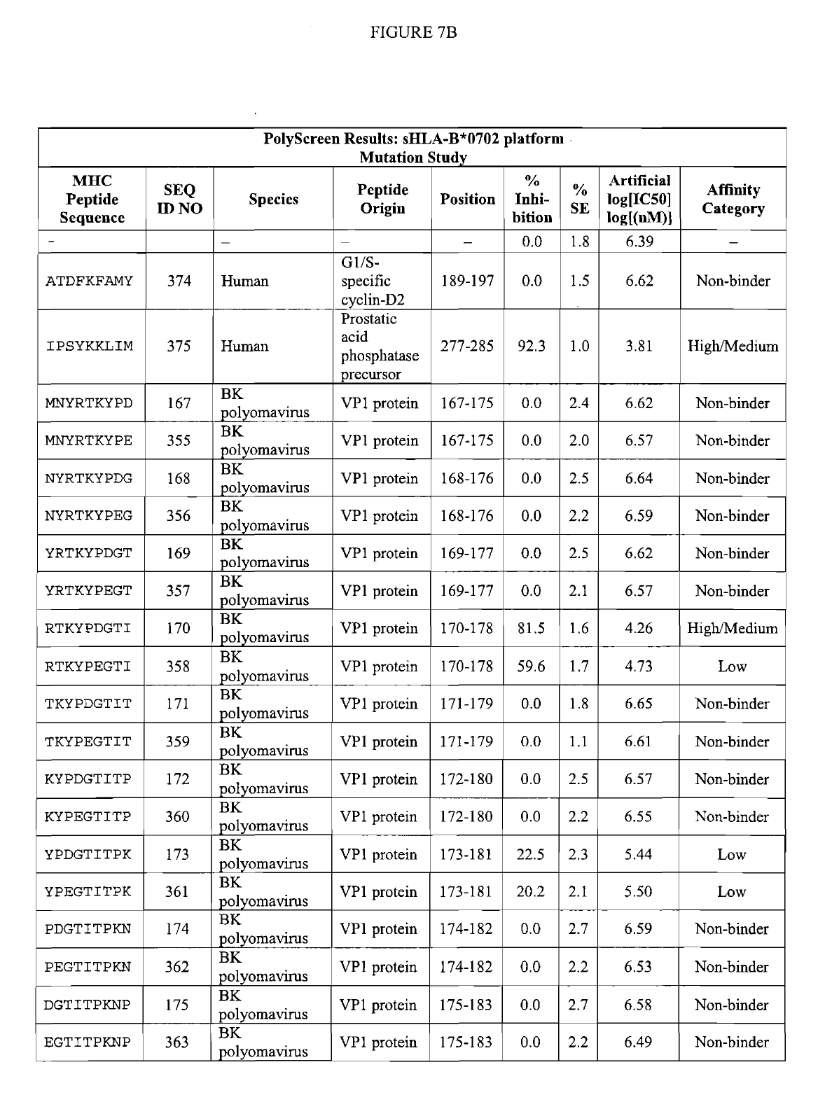

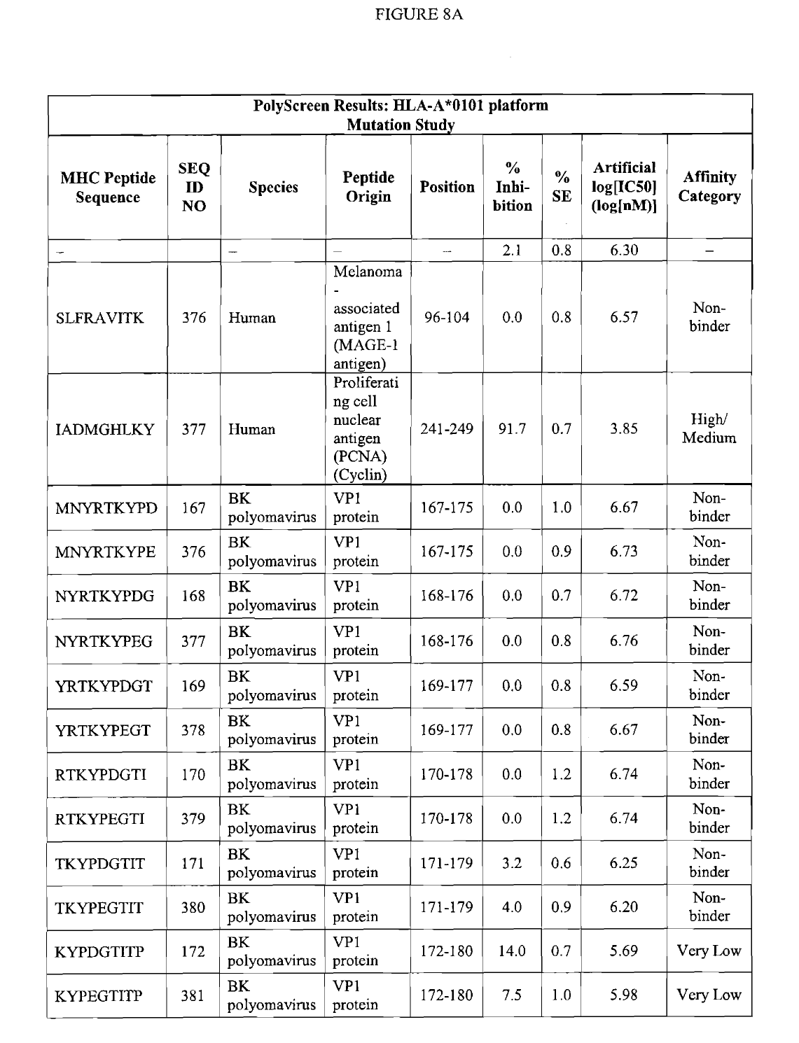

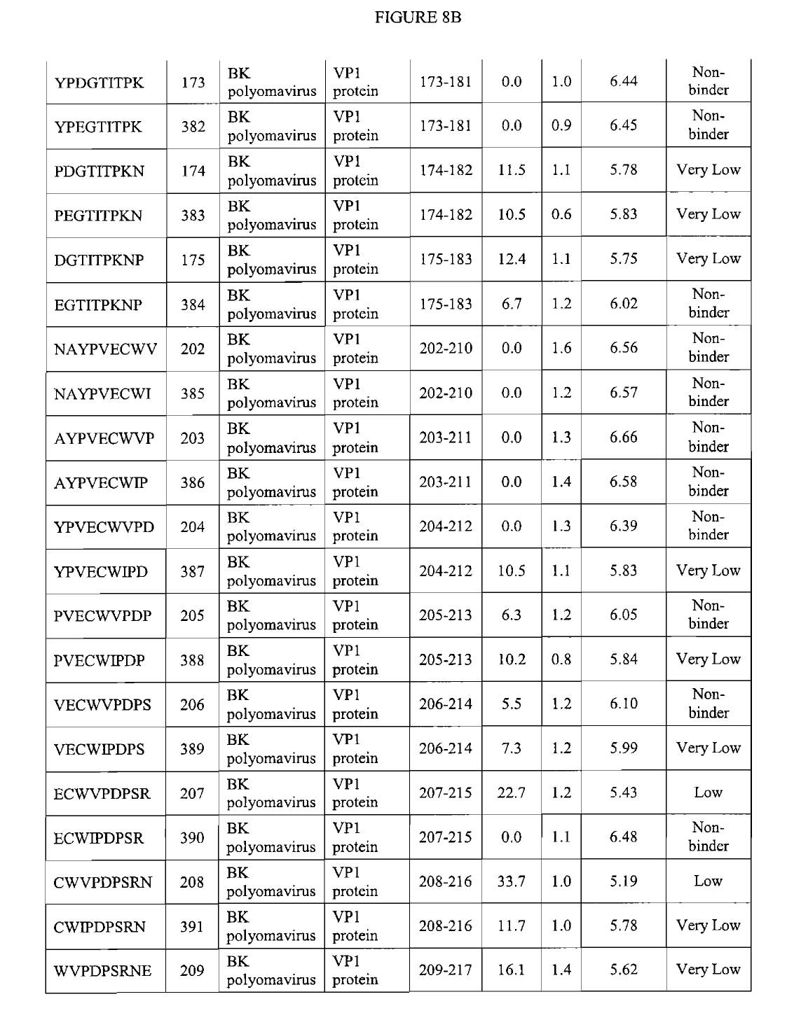

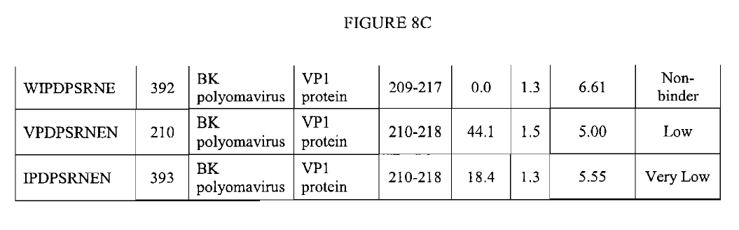

372 peptides representing all nine-amino acid peptides and 18 mutant nine-amino acid peptides of BKV VP1 (GenBank Accession No. AAT47405) were synthesized using standard techniques. Each peptide was evaluated with the PolyScreen sHLA-B*0702 and sHLA-A*0101 platforms (Pure Protein, L.L.C., Austin, Tex., USA) according to the manufacturer's instructions. In general, each peptide was evaluated in a competitive assay with a labeled reference peptide for binding with a recombinant soluble HLA molecule. The technique of fluorescence polarization was used to visualize such binding events. Some peptides identified as being high/medium binders using the PolyScreen platforms were tested using the PolyTest sHLA-B*0702 and sHLA-A*0101 (Pure Protein, L.L.C., Austin, Tex., USA) according to the manufacturer's instructions to determine actual binding affinity. Sequences, locations in the BKV VP1 polypeptide, sequence identifiers, the percent inhibition of the labeled reference peptide (% inhibition), the percent standard error of inhibition (% SE), estimated affinities, and actual affinities for peptides having an estimated IC₅₀ of less than 1 mM for HLA-B7 and HLA-A1 are listed in FIGS. 1 and 4, respectively. Data for peptides having an estimated IC₅₀ of greater than 1 mM for HLA-B7 are listed in FIGS. 2 and 3, and data for peptides having an estimated IC₅₀ of greater than 1 mM for HLA-A1 are listed in FIGS. 5 and 6. Sequences, locations in the BKV VP1 polypeptide, sequence identifiers, the percent inhibition of the labeled reference peptide (% inhibition), the percent standard error of inhibition (% SE), and estimated affinities of the 18 mutant nine-amino acid peptides of BKV VP1 for HLA-B7 and HLA-A1 are listed in FIGS. 7 and 8, respectively.

### Example 2

### Enumerating BKV-Specific CD8+ T Cells

Biotinylated MHC molecules are synthesized and folded with β2M and peptides from Table 1 or Table 2 using standard methods. See, e.g., Garboczi et al. (Proc. Nat. Acad. Sci., 89:3429-3433 (1992)), Sylvester-Hvid et al. (Tissue Antigens. 59(4):251 (2002)), U.S. Patent Application No. 20060035338, and U.S. Patent Application No. 20060134744. Biotinylated MHC molecules are attached to a labeled streptavidin molecule.

CD8+ T cells are obtained from whole blood. The CD8+ T cells are then incubated with the labeled BKV-MHC tetramers and labeled anti-IFN-gamma, anti-CD107a, and CD107b antibodies using standard procedures for labeling cells for flow cytometry. See, e.g., Appay and Rowland-Jones (J. Immunol. Methods. 268(1):9-19 (2002)), Kallas et al. (J. Infect. Dis. 179:1124-1131 (1999)), and Betts et al. (J. Immunol. Methods. 281(1-2):65-78 (2003)). The T cells are examined using flow cytometry to enumerate BKV-specific CD8+ T cells as well as assess their activation by IFN-gamma expression levels and cytolytic activity by CD107a and CD107b expression levels. BKV-antigen specific CD8+ T cell number, activation, and cytolytic activity from test patients are compared to BKV-antigen specific CD8+ T cell number, activation, and cytolytic activity from normal, healthy controls as well as renal transplant patients with and without BK-associated nephropathy, thereby providing information on the patient's immune competence to BKV.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated peptide selected from the group consisting of SEQ ID NOS:1-107, SEQ ID NOS:109-258, and SEQ ID NOS:260-373.

2. A composition comprising an MHC molecule and a peptide selected from the group consisting of SEQ ID NOS:1-107, SEQ ID NOS:109-258, and SEQ ID NOS:260-373.

3. The composition of claim 2, wherein said MHC molecule is selected from the group consisting of HLA-B7, HLA-A1, and HLA-A2.

4. The composition of claim 2, wherein said MHC molecule is selected from the group consisting of HLA-B8 and HLA-B35.

5. The composition of claim 2, wherein said peptide is selected from the group consisting of SEQ ID NO:342, SEQ ID NO:252, SEQ ID NO:2, SEQ ID NO:80, SEQ ID NO:14, and SEQ ID NO:182.

6. A composition comprising an MHC molecule and a peptide selected from the group consisting of SEQ ID NOS:1-373, wherein said MHC molecule is selected from the group consisting of HLA-B7 and HLA-A1.

7. A composition comprising an MHC/peptide multimer and a peptide selected from the group consisting of SEQ ID NOS:1-373.

8. The composition of claim 7, wherein said MHC molecule is selected from the group consisting of HLA-B7, HLA-A1, and HLA-A2.

9. The composition of claim 7, wherein said MHC molecule is selected from the group consisting of HLA-B8 and HLA-B35.

10. The composition of claim 7, wherein said peptide is selected from the group consisting of SEQ ID NO:342, SEQ ID NO:252, SEQ ID NO:2, SEQ ID NO:80, SEQ ID NO: 14, SEQ ID NO: 182, SEQ ID NO: 108, and SEQ ID NO:259.

11. The composition of claim 7, wherein said composition comprises more than one different peptide.

12. The composition of claim 11, wherein said composition comprises 2 different peptides.

13. The composition of claim 11, wherein the composition comprises 3 different peptides.

14. The composition of claim 11, wherein the composition comprises 4 different peptides.

15. The composition of claim 11, wherein the composition comprises more than 4 different peptides.

16. A kit comprising a peptide selected from the group consisting of SEQ ID NOS:1-373 and an antibody selected from the group consisting of anti-interferon-gamma, anti-TNF-alpha, anti-IL-2, anti-CD107a, and anti-CD107b.

17. A method of evaluating whether or not a subject is BK Virus immunocompetent, comprising contacting CD8+ T cells from said subject with a composition comprising a peptide selected from the group consisting of SEQ ID NOS:1-373, and evaluating one or more of said subject's BK Virus-specific CD8+ T cell parameters as compared to the minimum BK Virus-specific CD8+ T cell parameters required for BK Virus immunocompetence.

18. The method of claim 17, wherein said peptide is part of an MHC/peptide complex.

19. The method of claim 17, wherein said peptide is part of an MHC/peptide multimer.

20. The method of claim 19, wherein said evaluating comprises performing flow cytometry.

21. The method of claim 19, wherein said evaluating comprises performing an ELISA-based assay.

22. A method of treating BK Virus nephropathy in a transplant patient comprising evaluating whether said patient is BK Virus immunocompetent or immune incompetent, and administering an appropriate dose of an immunosuppressive drug.

23. The method of claim 22, wherein said immunosuppressive drug is tacrolimus.

24. The method of claim 22, wherein said immunosuppressive drug is rapamycin.

25. A method of treating BK Virus nephropathy in a transplant patient comprising determining whether said patient is BK Virus immune incompetent, and based on said determination of immunodeficiency, administering an appropriate dose of an antiviral drug.

26. The method of claim 25, wherein said antiviral drug is cidifovir.


SEQUENCE LISTING (377 sequences)

SEQ ID NO: 1 - protein, 9 aa, BK polyomavirus
MAPTKRKGE

SEQ ID NO: 2 - protein, 9 aa, BK polyomavirus
APTKRKGEC

SEQ ID NO: 3 - protein, 9 aa, BK polyomavirus
PTKRKGECP

SEQ ID NO: 4 - protein, 9 aa, BK polyomavirus
TKRKGECPG

SEQ ID NO: 5 - protein, 9 aa, BK polyomavirus
KRKGECPGA

SEQ ID NO: 6 - protein, 9 aa, BK polyomavirus
RKGECPGAA

SEQ ID NO: 7 - protein, 9 aa, BK polyomavirus
KGECPGAAP

SEQ ID NO: 8 - protein, 9 aa, BK polyomavirus
GECPGAAPK

SEQ ID NO: 9 - protein, 9 aa, BK polyomavirus
ECPGAAPKK

SEQ ID NO: 10 - protein, 9 aa, BK polyomavirus
CPGAAPKKP

SEQ ID NO: 11 - protein, 9 aa, BK polyomavirus
PGAAPKKPK

SEQ ID NO: 12 - protein, 9 aa, BK polyomavirus
GAAPKKPKE

SEQ ID NO: 13 - protein, 9 aa, BK polyomavirus
AAPKKPKEP

SEQ ID NO: 14 - protein, 9 aa, BK polyomavirus
APKKPKEPV

SEQ ID NO: 15 - protein, 9 aa, BK polyomavirus
PKKPKEPVQ

SEQ ID NO: 16 - protein, 9 aa, BK polyomavirus
KKPKEPVQV

SEQ ID NO: 17 - protein, 9 aa, BK polyomavirus
KPKEPVQVP

SEQ ID NO: 18 - protein, 9 aa, BK polyomavirus
PKEPVQVPK

SEQ ID NO: 19 - protein, 9 aa, BK polyomavirus
KEPVQVPKL

SEQ ID NO: 20 - protein, 9 aa, BK polyomavirus
EPVQVPKLL

SEQ ID NO: 21 - protein, 9 aa, BK polyomavirus
PVQVPKLLI

SEQ ID NO: 22 - protein, 9 aa, BK polyomavirus
VQVPKLLIK

SEQ ID NO: 23 - protein, 9 aa, BK polyomavirus
QVPKLLIKG

SEQ ID NO: 24 - protein, 9 aa, BK polyomavirus
VPKLLIKGG

SEQ ID NO: 25 - protein, 9 aa, BK polyomavirus
PKLLIKGGV

SEQ ID NO: 26 - protein, 9 aa, BK polyomavirus
KLLIKGGVE

SEQ ID NO: 27 - protein, 9 aa, BK polyomavirus
LLIKGGVEV

SEQ ID NO: 28 - protein, 9 aa, BK polyomavirus
LIKGGVEVL

SEQ ID NO: 29 - protein, 9 aa, BK polyomavirus
IKGGVEVLE

SEQ ID NO: 30 - protein, 9 aa, BK polyomavirus
KGGVEVLEV

SEQ ID NO: 31 - protein, 9 aa, BK polyomavirus
GGVEVLEVK

SEQ ID NO: 32 - protein, 9 aa, BK polyomavirus
GVEVLEVKT

SEQ ID NO: 33 - protein, 9 aa, BK polyomavirus
VEVLEVKTG

SEQ ID NO: 34 - protein, 9 aa, BK polyomavirus
EVLEVKTGV

SEQ ID NO: 35 - protein, 9 aa, BK polyomavirus
VLEVKTGVD

SEQ ID NO: 36 - protein, 9 aa, BK polyomavirus
LEVKTGVDA

SEQ ID NO: 37 - protein, 9 aa, BK polyomavirus
EVKTGVDAI

SEQ ID NO: 38 - protein, 9 aa, BK polyomavirus
VKTGVDAIT

SEQ ID NO: 39 - protein, 9 aa, BK polyomavirus
KTGVDAITE

SEQ ID NO: 40 - protein, 9 aa, BK polyomavirus
TGVDAITEV

SEQ ID NO: 41 - protein, 9 aa, BK polyomavirus
GVDAITEVE

SEQ ID NO: 42 - protein, 9 aa, BK polyomavirus
VDAITEVEC

SEQ ID NO: 43 - protein, 9 aa, BK polyomavirus
DAITEVECF

SEQ ID NO: 44 - protein, 9 aa, BK polyomavirus
AITEVECFL

SEQ ID NO: 45 - protein, 9 aa, BK polyomavirus
ITEVECFLN

SEQ ID NO: 46 - protein, 9 aa, BK polyomavirus
TEVECFLNP

SEQ ID NO: 47 - protein, 9 aa, BK polyomavirus
EVECFLNPE

SEQ ID NO: 48 - protein, 9 aa, BK polyomavirus
VECFLNPEM

SEQ ID NO: 49 - protein, 9 aa, BK polyomavirus
ECFLNPEMG

SEQ ID NO: 50 - protein, 9 aa, BK polyomavirus
CFLNPEMGD

SEQ ID NO: 51 - protein, 9 aa, BK polyomavirus
FLNPEMGDP

SEQ ID NO: 52 - protein, 9 aa, BK polyomavirus
LNPEMGDPD

SEQ ID NO: 53 - protein, 9 aa, BK polyomavirus
NPEMGDPDE

SEQ ID NO: 54 - protein, 9 aa, BK polyomavirus
PEMGDPDEN

SEQ ID NO: 55 - protein, 9 aa, BK polyomavirus
EMGDPDENL

SEQ ID NO: 56 - protein, 9 aa, BK polyomavirus
MGDPDENLR

SEQ ID NO: 57 - protein, 9 aa, BK polyomavirus
GDPDENLRG

SEQ ID NO: 58 - protein, 9 aa, BK polyomavirus
DPDENLRGF

SEQ ID NO: 59 - protein, 9 aa, BK polyomavirus
PDENLRGFS

SEQ ID NO: 60 - protein, 9 aa, BK polyomavirus
DENLRGFSL

SEQ ID NO: 61 - protein, 9 aa, BK polyomavirus
ENLRGFSLK

SEQ ID NO: 62 - protein, 9 aa, BK polyomavirus
NLRGFSLKL

SEQ ID NO: 63 - protein, 9 aa, BK polyomavirus
LRGFSLKLS

SEQ ID NO: 64 - protein, 9 aa, BK polyomavirus
RGFSLKLSA

SEQ ID NO: 65 - protein, 9 aa, BK polyomavirus
GFSLKLSAE

SEQ ID NO: 66 - protein, 9 aa, BK polyomavirus
FSLKLSAEN

SEQ ID NO: 67 - protein, 9 aa, BK polyomavirus
SLKLSAEND

SEQ ID NO: 68 - protein, 9 aa, BK polyomavirus
LKLSAENDF

SEQ ID NO: 69 - protein, 9 aa, BK polyomavirus
KLSAENDFS

SEQ ID NO: 70 - protein, 9 aa, BK polyomavirus
LSAENDFSS

SEQ ID NO: 71 - protein, 9 aa, BK polyomavirus
SAENDFSSD

SEQ ID NO: 72 - protein, 9 aa, BK polyomavirus
AENDFSSDS

SEQ ID NO: 73 - protein, 9 aa, BK polyomavirus
ENDFSSDSP

SEQ ID NO: 74 - protein, 9 aa, BK polyomavirus
NDFSSDSPE

SEQ ID NO: 75 - protein, 9 aa, BK polyomavirus
DFSSDSPER

SEQ ID NO: 76 - protein, 9 aa, BK polyomavirus
FSSDSPERK

SEQ ID NO: 77 - protein, 9 aa, BK polyomavirus
SSDSPERKM

SEQ ID NO: 78 - protein, 9 aa, BK polyomavirus
SDSPERKML

SEQ ID NO: 79 - protein, 9 aa, BK polyomavirus
DSPERKMLP

SEQ ID NO: 80 - protein, 9 aa, BK polyomavirus
SPERKMLPC

SEQ ID NO: 81 - protein, 9 aa, BK polyomavirus
PERKMLPCY

SEQ ID NO: 82 - protein, 9 aa, BK polyomavirus
ERKMLPCYS

SEQ ID NO: 83 - protein, 9 aa, BK polyomavirus
RKMLPCYST

SEQ ID NO: 84 - protein, 9 aa, BK polyomavirus
KMLPCYSTA

SEQ ID NO: 85 - protein, 9 aa, BK polyomavirus
MLPCYSTAR

SEQ ID NO: 86 - protein, 9 aa, BK polyomavirus
LPCYSTARI

SEQ ID NO: 87 - protein, 9 aa, BK polyomavirus
PCYSTARIP

SEQ ID NO: 88 - protein, 9 aa, BK polyomavirus
CYSTARIPL

SEQ ID NO: 89 - protein, 9 aa, BK polyomavirus
YSTARIPLP

SEQ ID NO: 90 - protein, 9 aa, BK polyomavirus
STARIPLPN

SEQ ID NO: 91 - protein, 9 aa, BK polyomavirus
TARIPLPNL

SEQ ID NO: 92 - protein, 9 aa, BK polyomavirus
ARIPLPNLN

SEQ ID NO: 93 - protein, 9 aa, BK polyomavirus
RIPLPNLNE

SEQ ID NO: 94 - protein, 9 aa, BK polyomavirus
IPLPNLNED

SEQ ID NO: 95 - protein, 9 aa, BK polyomavirus
PLPNLNEDL

SEQ ID NO: 96 - protein, 9 aa, BK polyomavirus
LPNLNEDLT

SEQ ID NO: 97 - protein, 9 aa, BK polyomavirus
PNLNEDLTC

SEQ ID NO: 98 - protein, 9 aa, BK polyomavirus
NLNEDLTCG

SEQ ID NO: 99 - protein, 9 aa, BK polyomavirus
LNEDLTCGN

SEQ ID NO: 100 - protein, 9 aa, BK polyomavirus
NEDLTCGNL

SEQ ID NO: 101 - protein, 9 aa, BK polyomavirus
EDLTCGNLL

SEQ ID NO: 102 - protein, 9 aa, BK polyomavirus
DLTCGNLLM

SEQ ID NO: 103 - protein, 9 aa, BK polyomavirus
LTCGNLLMW

SEQ ID NO: 104 - protein, 9 aa, BK polyomavirus
TCGNLLMWE

SEQ ID NO: 105 - protein, 9 aa, BK polyomavirus
CGNLLMWEA

SEQ ID NO: 106 - protein, 9 aa, BK polyomavirus
GNLLMWEAV

SEQ ID NO: 107 - protein, 9 aa, BK polyomavirus
NLLMWEAVT

SEQ ID NO: 108 - protein, 9 aa, BK polyomavirus
LLMWEAVTV

SEQ ID NO: 109 - protein, 9 aa, BK polyomavirus
LMWEAVTVQ

SEQ ID NO: 110 - protein, 9 aa, BK polyomavirus
MWEAVTVQT

SEQ ID NO: 111 - protein, 9 aa, BK polyomavirus
WEAVTVQTE

SEQ ID NO: 112 - protein, 9 aa, BK polyomavirus
EAVTVQTEV

SEQ ID NO: 113 - protein, 9 aa, BK polyomavirus
AVTVQTEVI

SEQ ID NO: 114 - protein, 9 aa, BK polyomavirus
VTVQTEVIG

SEQ ID NO: 115 - protein, 9 aa, BK polyomavirus
TVQTEVIGI

SEQ ID NO: 116 - protein, 9 aa, BK polyomavirus
VQTEVIGIT

SEQ ID NO: 117 - protein, 9 aa, BK polyomavirus
QTEVIGITS

SEQ ID NO: 118 - protein, 9 aa, BK polyomavirus
TEVIGITSM

SEQ ID NO: 119 - protein, 9 aa, BK polyomavirus
EVIGITSML

SEQ ID NO: 120 - protein, 9 aa, BK polyomavirus
VIGITSMLN

SEQ ID NO: 121 - protein, 9 aa, BK polyomavirus
IGITSMLNL

SEQ ID NO: 122 - protein, 9 aa, BK polyomavirus
GITSMLNLH

SEQ ID NO: 123 - protein, 9 aa, BK polyomavirus
ITSMLNLHA

SEQ ID NO: 124 - protein, 9 aa, BK polyomavirus
TSMLNLHAG

SEQ ID NO: 125 - protein, 9 aa, BK polyomavirus
SMLNLHAGS

SEQ ID NO: 126 - protein, 9 aa, BK polyomavirus
MLNLHAGSQ

SEQ ID NO: 127 - protein, 9 aa, BK polyomavirus
LNLHAGSQK

SEQ ID NO: 128 - protein, 9 aa, BK polyomavirus
NLHAGSQKV

SEQ ID NO: 129 - protein, 9 aa, BK polyomavirus
LHAGSQKVH

SEQ ID NO: 130 - protein, 9 aa, BK polyomavirus
HAGSQKVHE

SEQ ID NO: 131 - protein, 9 aa, BK polyomavirus
AGSQKVHEH

SEQ ID NO: 132 - protein, 9 aa, BK polyomavirus
GSQKVHEHG

SEQ ID NO: 133 - protein, 9 aa, BK polyomavirus
SQKVHEHGG

SEQ ID NO: 134 - protein, 9 aa, BK polyomavirus
QKVHEHGGG

SEQ ID NO: 135 - protein, 9 aa, BK polyomavirus
KVHEHGGGK

SEQ ID NO: 136 - protein, 9 aa, BK polyomavirus
VHEHGGGKP

SEQ ID NO: 137 - protein, 9 aa, BK polyomavirus
HEHGGGKPI

SEQ ID NO: 138 - protein, 9 aa, BK polyomavirus
EHGGGKPIQ

SEQ ID NO: 139 - protein, 9 aa, BK polyomavirus
HGGGKPIQG

SEQ ID NO: 140 - protein, 9 aa, BK polyomavirus
GGGKPIQGS

SEQ ID NO: 141 - protein, 9 aa, BK polyomavirus
GGKPIQGSN

SEQ ID NO: 142 - protein, 9 aa, BK polyomavirus
GKPIQGSNF

SEQ ID NO: 143 - protein, 9 aa, BK polyomavirus
KPIQGSNFH

SEQ ID NO: 144 - protein, 9 aa, BK polyomavirus
PIQGSNFHF

SEQ ID NO: 145 - protein, 9 aa, BK polyomavirus
IQGSNFHFF

SEQ ID NO: 146 - protein, 9 aa, BK polyomavirus
QGSNFHFFA

SEQ ID NO: 147 - protein, 9 aa, BK polyomavirus
GSNFHFFAV

SEQ ID NO: 148 - protein, 9 aa, BK polyomavirus
SNFHFFAVG

SEQ ID NO: 149 - protein, 9 aa, BK polyomavirus
NFHFFAVGG

SEQ ID NO: 150 - protein, 9 aa, BK polyomavirus
FHFFAVGGD

SEQ ID NO: 151 - protein, 9 aa, BK polyomavirus
HFFAVGGDP

SEQ ID NO: 152 - protein, 9 aa, BK polyomavirus
FFAVGGDPL

SEQ ID NO: 153 - protein, 9 aa, BK polyomavirus
FAVGGDPLE

SEQ ID NO: 154 - protein, 9 aa, BK polyomavirus
AVGGDPLEM

SEQ ID NO: 155 - protein, 9 aa, BK polyomavirus
VGGDPLEMQ

SEQ ID NO: 156 - protein, 9 aa, BK polyomavirus
GGDPLEMQG

SEQ ID NO: 157 - protein, 9 aa, BK polyomavirus
GDPLEMQGV

SEQ ID NO: 158 - protein, 9 aa, BK polyomavirus
DPLEMQGVL

SEQ ID NO: 159 - protein, 9 aa, BK polyomavirus
PLEMQGVLM

SEQ ID NO: 160 - protein, 9 aa, BK polyomavirus
LEMQGVLMN

SEQ ID NO: 161 - protein, 9 aa, BK polyomavirus
EMQGVLMNY

SEQ ID NO: 162 - protein, 9 aa, BK polyomavirus
MQGVLMNYR

SEQ ID NO: 163 - protein, 9 aa, BK polyomavirus
QGVLMNYRT

SEQ ID NO: 164 - protein, 9 aa, BK polyomavirus
GVLMNYRTK

SEQ ID NO: 165 - protein, 9 aa, BK polyomavirus
VLMNYRTKY

SEQ ID NO: 166 - protein, 9 aa, BK polyomavirus
LMNYRTKYP

SEQ ID NO: 167 - protein, 9 aa, BK polyomavirus
MNYRTKYPD

SEQ ID NO: 168 - protein, 9 aa, BK polyomavirus
NYRTKYPDG

SEQ ID NO: 169 - protein, 9 aa, BK polyomavirus
YRTKYPDGT

SEQ ID NO: 170 - protein, 9 aa, BK polyomavirus
RTKYPDGTI

SEQ ID NO: 171 - protein, 9 aa, BK polyomavirus
TKYPDGTIT

SEQ ID NO: 172 - protein, 9 aa, BK polyomavirus
KYPDGTITP

SEQ ID NO: 173 - protein, 9 aa, BK polyomavirus
YPDGTITPK

SEQ ID NO: 174 - protein, 9 aa, BK polyomavirus
PDGTITPKN

SEQ ID NO: 175 - protein, 9 aa, BK polyomavirus
DGTITPKNP

SEQ ID NO: 176 - protein, 9 aa, BK polyomavirus
GTITPKNPT

SEQ ID NO: 177 - protein, 9 aa, BK polyomavirus
TITPKNPTA

SEQ ID NO: 178 - protein, 9 aa, BK polyomavirus
ITPKNPTAQ

SEQ ID NO: 179 - protein, 9 aa, BK polyomavirus
TPKNPTAQS

SEQ ID NO: 180 - protein, 9 aa, BK polyomavirus
PKNPTAQSQ

SEQ ID NO: 181 - protein, 9 aa, BK polyomavirus
KNPTAQSQV

SEQ ID NO: 182 - protein, 9 aa, BK polyomavirus
NPTAQSQVM

SEQ ID NO: 183 - protein, 9 aa, BK polyomavirus
PTAQSQVMN

SEQ ID NO: 184 - protein, 9 aa, BK polyomavirus
TAQSQVMNT

SEQ ID NO: 185 - protein, 9 aa, BK polyomavirus
AQSQVMNTD

SEQ ID NO: 186 - protein, 9 aa, BK polyomavirus
QSQVMNTDH

SEQ ID NO: 187 - protein, 9 aa, BK polyomavirus
SQVMNTDHK

SEQ ID NO: 188 - protein, 9 aa, BK polyomavirus
QVMNTDHKA

SEQ ID NO: 189 - protein, 9 aa, BK polyomavirus
VMNTDHKAY

SEQ ID NO: 190 - protein, 9 aa, BK polyomavirus
MNTDHKAYL

SEQ ID NO: 191 - protein, 9 aa, BK polyomavirus
NTDHKAYLD

SEQ ID NO: 192 - protein, 9 aa, BK polyomavirus
TDHKAYLDK

SEQ ID NO: 193 - protein, 9 aa, BK polyomavirus
DHKAYLDKN

SEQ ID NO: 194 - protein, 9 aa, BK polyomavirus
HKAYLDKNN

SEQ ID NO: 195 - protein, 9 aa, BK polyomavirus
KAYLDKNNA

SEQ ID NO: 196 - protein, 9 aa, BK polyomavirus
AYLDKNNAY

SEQ ID NO: 197 - protein, 9 aa, BK polyomavirus
YLDKNNAYP

SEQ ID NO: 198 - protein, 9 aa, BK polyomavirus
LDKNNAYPV

SEQ ID NO: 199 - protein, 9 aa, BK polyomavirus
DKNNAYPVE

SEQ ID NO: 200 - protein, 9 aa, BK polyomavirus
KNNAYPVEC

SEQ ID NO: 201 - protein, 9 aa, BK polyomavirus
NNAYPVECW

SEQ ID NO: 202 - protein, 9 aa, BK polyomavirus
NAYPVECWV

SEQ ID NO: 203 - protein, 9 aa, BK polyomavirus
AYPVECWVP

SEQ ID NO: 204 - protein, 9 aa, BK polyomavirus
YPVECWVPD

SEQ ID NO: 205 - protein, 9 aa, BK polyomavirus
PVECWVPDP

SEQ ID NO: 206 - protein, 9 aa, BK polyomavirus
VECWVPDPS

SEQ ID NO: 207 - protein, 9 aa, BK polyomavirus
ECWVPDPSR

SEQ ID NO: 208 - protein, 9 aa, BK polyomavirus
CWVPDPSRN

SEQ ID NO: 209 - protein, 9 aa, BK polyomavirus
WVPDPSRNE

SEQ ID NO: 210 - protein, 9 aa, BK polyomavirus
VPDPSRNEN

SEQ ID NO: 211 - protein, 9 aa, BK polyomavirus
PDPSRNENT

SEQ ID NO: 212 - protein, 9 aa, BK polyomavirus
DPSRNENTR

SEQ ID NO: 213 - protein, 9 aa, BK polyomavirus
PSRNENTRY

SEQ ID NO: 214 - protein, 9 aa, BK polyomavirus
SRNENTRYF

SEQ ID NO: 215 - protein, 9 aa, BK polyomavirus
RNENTRYFG

SEQ ID NO: 216 - protein, 9 aa, BK polyomavirus
NENTRYFGT

SEQ ID NO: 217 - protein, 9 aa, BK polyomavirus
ENTRYFGTF

SEQ ID NO: 218 - protein, 9 aa, BK polyomavirus
NTRYFGTFT

SEQ ID NO: 219 - protein, 9 aa, BK polyomavirus
TRYFGTFTG

SEQ ID NO: 220 - protein, 9 aa, BK polyomavirus
RYFGTFTGG

SEQ ID NO: 221 - protein, 9 aa, BK polyomavirus
YFGTFTGGE

SEQ ID NO: 222 - protein, 9 aa, BK polyomavirus
FGTFTGGEN

SEQ ID NO: 223 - protein, 9 aa, BK polyomavirus
GTFTGGENV

SEQ ID NO: 224 - protein, 9 aa, BK polyomavirus
TFTGGENVP

SEQ ID NO: 225 - protein, 9 aa, BK polyomavirus
FTGGENVPP

SEQ ID NO: 226 - protein, 9 aa, BK polyomavirus
TGGENVPPV

SEQ ID NO: 227 - protein, 9 aa, BK polyomavirus
GGENVPPVL

SEQ ID NO: 228 - protein, 9 aa, BK polyomavirus
GENVPPVLH

SEQ ID NO: 229 - protein, 9 aa, BK polyomavirus
ENVPPVLHV

SEQ ID NO: 230 - protein, 9 aa, BK polyomavirus
NVPPVLHVT

SEQ ID NO: 231 - protein, 9 aa, BK polyomavirus
VPPVLHVTN

SEQ ID NO: 232 - protein, 9 aa, BK polyomavirus
PPVLHVTNT

SEQ ID NO: 233 - protein, 9 aa, BK polyomavirus
PVLHVTNTA

SEQ ID NO: 234 - protein, 9 aa, BK polyomavirus
VLHVTNTAT

SEQ ID NO: 235 - protein, 9 aa, BK polyomavirus
LHVTNTATT

SEQ ID NO: 236 - protein, 9 aa, BK polyomavirus
HVTNTATTV

SEQ ID NO: 237 - protein, 9 aa, BK polyomavirus
VTNTATTVL

SEQ ID NO: 238 - protein, 9 aa, BK polyomavirus
TNTATTVLL

SEQ ID NO: 239 - protein, 9 aa, BK polyomavirus
NTATTVLLD

SEQ ID NO: 240 - protein, 9 aa, BK polyomavirus
TATTVLLDE

SEQ ID NO: 241 - protein, 9 aa, BK polyomavirus
ATTVLLDEQ

SEQ ID NO: 242 - protein, 9 aa, BK polyomavirus
TTVLLDEQG

SEQ ID NO: 243 - protein, 9 aa, BK polyomavirus
TVLLDEQGV

SEQ ID NO: 244 - protein, 9 aa, BK polyomavirus
VLLDEQGVG

SEQ ID NO: 245 - protein, 9 aa, BK polyomavirus
LLDEQGVGP

SEQ ID NO: 246 - protein, 9 aa, BK polyomavirus
LDEQGVGPL

SEQ ID NO: 247 - protein, 9 aa, BK polyomavirus
DEQGVGPLC

SEQ ID NO: 248 - protein, 9 aa, BK polyomavirus
EQGVGPLCK

SEQ ID NO: 249 - protein, 9 aa, BK polyomavirus
QGVGPLCKA

SEQ ID NO: 250 - protein, 9 aa, BK polyomavirus
GVGPLCKAD

SEQ ID NO: 251 - protein, 9 aa, BK polyomavirus
VGPLCKADS

SEQ ID NO: 252 - protein, 9 aa, BK polyomavirus
GPLCKADSL

SEQ ID NO: 253 - protein, 9 aa, BK polyomavirus
PLCKADSLY

SEQ ID NO: 254 - protein, 9 aa, BK polyomavirus
LCKADSLYV

SEQ ID NO: 255 - protein, 9 aa, BK polyomavirus
CKADSLYVS

SEQ ID NO: 256 - protein, 9 aa, BK polyomavirus
KADSLYVSA

SEQ ID NO: 257 - protein, 9 aa, BK polyomavirus
ADSLYVSAA

SEQ ID NO: 258 - protein, 9 aa, BK polyomavirus
DSLYVSAAD

SEQ ID NO: 259 - protein, 9 aa, BK polyomavirus
SLYVSAADI

SEQ ID NO: 260 - protein, 9 aa, BK polyomavirus
LYVSAADIC

SEQ ID NO: 261 - protein, 9 aa, BK polyomavirus
YVSAADICG

SEQ ID NO: 262 - protein, 9 aa, BK polyomavirus
VSAADICGL

SEQ ID NO: 263 - protein, 9 aa, BK polyomavirus
SAADICGLF

SEQ ID NO: 264 - protein, 9 aa, BK polyomavirus
AADICGLFT

SEQ ID NO: 265 - protein, 9 aa, BK polyomavirus
ADICGLFTN

SEQ ID NO: 266 - protein, 9 aa, BK polyomavirus
DICGLFTNS

SEQ ID NO: 267 - protein, 9 aa, BK polyomavirus
ICGLFTNSS

SEQ ID NO: 268 - protein, 9 aa, BK polyomavirus
CGLFTNSSG

SEQ ID NO: 269 - protein, 9 aa, BK polyomavirus
GLFTNSSGT

SEQ ID NO: 270 - protein, 9 aa, BK polyomavirus
LFTNSSGTQ

SEQ ID NO: 271 - protein, 9 aa, BK polyomavirus
FTNSSGTQQ

SEQ ID NO: 272 - protein, 9 aa, BK polyomavirus
TNSSGTQQW

SEQ ID NO: 273 - protein, 9 aa, BK polyomavirus
NSSGTQQWR

SEQ ID NO: 274 - protein, 9 aa, BK polyomavirus
SSGTQQWRG

SEQ ID NO: 275 - protein, 9 aa, BK polyomavirus
SGTQQWRGL

SEQ ID NO: 276 - protein, 9 aa, BK polyomavirus
GTQQWRGLA

SEQ ID NO: 277 - protein, 9 aa, BK polyomavirus
TQQWRGLAR

SEQ ID NO: 278 - protein, 9 aa, BK polyomavirus
QQWRGLARY

SEQ ID NO: 279 - protein, 9 aa, BK polyomavirus
QWRGLARYF

SEQ ID NO: 280 - protein, 9 aa, BK polyomavirus
WRGLARYFK

SEQ ID NO: 281 - protein, 9 aa, BK polyomavirus
RGLARYFKI

SEQ ID NO: 282 - protein, 9 aa, BK polyomavirus
GLARYFKIR

SEQ ID NO: 283 - protein, 9 aa, BK polyomavirus
LARYFKIRL

SEQ ID NO: 284 - protein, 9 aa, BK polyomavirus
ARYFKIRLR

SEQ ID NO: 285 - protein, 9 aa, BK polyomavirus
RYFKIRLRK

SEQ ID NO: 286 - protein, 9 aa, BK polyomavirus
YFKIRLRKR

SEQ ID NO: 287 - protein, 9 aa, BK polyomavirus
FKIRLRKRS

SEQ ID NO: 288 - protein, 9 aa, BK polyomavirus
KIRLRKRSV

SEQ ID NO: 289 - protein, 9 aa, BK polyomavirus
IRLRKRSVK

SEQ ID NO: 290 - protein, 9 aa, BK polyomavirus
RLRKRSVKN

SEQ ID NO: 291 - protein, 9 aa, BK polyomavirus
LRKRSVKNP

SEQ ID NO: 292 - protein, 9 aa, BK polyomavirus
RKRSVKNPY

SEQ ID NO: 293 - protein, 9 aa, BK polyomavirus
KRSVKNPYP

SEQ ID NO: 294 - protein, 9 aa, BK polyomavirus
RSVKNPYPI

SEQ ID NO: 295 - protein, 9 aa, BK polyomavirus
SVKNPYPIS

SEQ ID NO: 296 - protein, 9 aa, BK polyomavirus
VKNPYPISF

SEQ ID NO: 297 - protein, 9 aa, BK polyomavirus
KNPYPISFL

SEQ ID NO: 298 - protein, 9 aa, BK polyomavirus
NPYPISFLL

SEQ ID NO: 299 - protein, 9 aa, BK polyomavirus
PYPISFLLS

SEQ ID NO: 300 - protein, 9 aa, BK polyomavirus
YPISFLLSD

SEQ ID NO: 301 - protein, 9 aa, BK polyomavirus
PISFLLSDL

SEQ ID NO: 302 - protein, 9 aa, BK polyomavirus
ISFLLSDLI

SEQ ID NO: 303 - protein, 9 aa, BK polyomavirus
SFLLSDLIN

SEQ ID NO: 304 - protein, 9 aa, BK polyomavirus
FLLSDLINR

SEQ ID NO: 305 - protein, 9 aa, BK polyomavirus
LLSDLINRR

SEQ ID NO: 306 - protein, 9 aa, BK polyomavirus
LSDLINRRT

SEQ ID NO: 307 - protein, 9 aa, BK polyomavirus
SDLINRRTQ

SEQ ID NO: 308 - protein, 9 aa, BK polyomavirus
DLINRRTQR

SEQ ID NO: 309 - protein, 9 aa, BK polyomavirus
LINRRTQRV

SEQ ID NO: 310 - protein, 9 aa, BK polyomavirus
INRRTQRVD

SEQ ID NO: 311 - protein, 9 aa, BK polyomavirus
NRRTQRVDG

SEQ ID NO: 312 - protein, 9 aa, BK polyomavirus
RRTQRVDGQ

SEQ ID NO: 313 - protein, 9 aa, BK polyomavirus
RTQRVDGQP

SEQ ID NO: 314 - protein, 9 aa, BK polyomavirus
TQRVDGQPM

SEQ ID NO: 315 - protein, 9 aa, BK polyomavirus
QRVDGQPMY

SEQ ID NO: 316 - protein, 9 aa, BK polyomavirus
RVDGQPMYG

SEQ ID NO: 317 - protein, 9 aa, BK polyomavirus
VDGQPMYGM

SEQ ID NO: 318 - protein, 9 aa, BK polyomavirus
DGQPMYGME

SEQ ID NO: 319 - protein, 9 aa, BK polyomavirus
GQPMYGMES

SEQ ID NO: 320 - protein, 9 aa, BK polyomavirus
QPMYGMESQ

SEQ ID NO: 321 - protein, 9 aa, BK polyomavirus
PMYGMESQV

SEQ ID NO: 322 - protein, 9 aa, BK polyomavirus
MYGMESQVE

SEQ ID NO: 323 - protein, 9 aa, BK polyomavirus
YGMESQVEE

SEQ ID NO: 324 - protein, 9 aa, BK polyomavirus
GMESQVEEV

SEQ ID NO: 325 - protein, 9 aa, BK polyomavirus
MESQVEEVR

SEQ ID NO: 326 - protein, 9 aa, BK polyomavirus
ESQVEEVRV

SEQ ID NO: 327 - protein, 9 aa, BK polyomavirus
SQVEEVRVF

SEQ ID NO: 328 - protein, 9 aa, BK polyomavirus
QVEEVRVFD

SEQ ID NO: 329 - protein, 9 aa, BK polyomavirus
VEEVRVFDG

SEQ ID NO: 330 - protein, 9 aa, BK polyomavirus
EEVRVFDGT

SEQ ID NO: 331 - protein, 9 aa, BK polyomavirus
EVRVFDGTE

SEQ ID NO: 332 - protein, 9 aa, BK polyomavirus
VRVFDGTER

SEQ ID NO: 333 - protein, 9 aa, BK polyomavirus
RVFDGTERL

SEQ ID NO: 334 - protein, 9 aa, BK polyomavirus
VFDGTERLP

SEQ ID NO: 335 - protein, 9 aa, BK polyomavirus
FDGTERLPG

SEQ ID NO: 336 - protein, 9 aa, BK polyomavirus
DGTERLPGD

SEQ ID NO: 337 - protein, 9 aa, BK polyomavirus
GTERLPGDP

SEQ ID NO: 338 - protein, 9 aa, BK polyomavirus
TERLPGDPD

SEQ ID NO: 339 - protein, 9 aa, BK polyomavirus
ERLPGDPDM

SEQ ID NO: 340 - protein, 9 aa, BK polyomavirus
RLPGDPDMI

SEQ ID NO: 341 - protein, 9 aa, BK polyomavirus
LPGDPDMIR

SEQ ID NO: 342 - protein, 9 aa, BK polyomavirus
PGDPDMIRY

SEQ ID NO: 343 - protein, 9 aa, BK polyomavirus
GDPDMIRYI

SEQ ID NO: 344 - protein, 9 aa, BK polyomavirus
DPDMIRYID

SEQ ID NO: 345 - protein, 9 aa, BK polyomavirus
PDMIRYIDK

SEQ ID NO: 346 - protein, 9 aa, BK polyomavirus
DMIRYIDKQ

SEQ ID NO: 347 - protein, 9 aa, BK polyomavirus
MIRYIDKQG

SEQ ID NO: 348 - protein, 9 aa, BK polyomavirus
IRYIDKQGQ

SEQ ID NO: 349 - protein, 9 aa, BK polyomavirus
RYIDKQGQL

SEQ ID NO: 350 - protein, 9 aa, BK polyomavirus
YIDKQGQLQ

SEQ ID NO: 351 - protein, 9 aa, BK polyomavirus
IDKQGQLQT

SEQ ID NO: 352 - protein, 9 aa, BK polyomavirus
DKQGQLQTK

SEQ ID NO: 353 - protein, 9 aa, BK polyomavirus
KQGQLQTKM

SEQ ID NO: 354 - protein, 9 aa, BK polyomavirus
QGQLQTKML

SEQ ID NO: 355 - protein, 9 aa, BK polyomavirus
MNYRTKYPE

SEQ ID NO: 356 - protein, 9 aa, BK polyomavirus
NYRTKYPEG

SEQ ID NO: 357 - protein, 9 aa, BK polyomavirus
YRTKYPEGT

SEQ ID NO: 358 - protein, 9 aa, BK polyomavirus
RTKYPEGTI

SEQ ID NO: 359 - protein, 9 aa, BK polyomavirus
TKYPEGTIT

SEQ ID NO: 360 - protein, 9 aa, BK polyomavirus
KYPEGTITP

SEQ ID NO: 361 - protein, 9 aa, BK polyomavirus
YPEGTITPK

SEQ ID NO: 362 - protein, 9 aa, BK polyomavirus
PEGTITPKN

SEQ ID NO: 363 - protein, 9 aa, BK polyomavirus
EGTITPKNP

SEQ ID NO: 364 - protein, 9 aa, BK polyomavirus
NAYPVECWI

SEQ ID NO: 365 - protein, 9 aa, BK polyomavirus
AYPVECWIP

SEQ ID NO: 366 - protein, 9 aa, BK polyomavirus
YPVECWIPD

SEQ ID NO: 367 - protein, 9 aa, BK polyomavirus
PVECWIPDP

SEQ ID NO: 368 - protein, 9 aa, BK polyomavirus
VECWIPDPS

SEQ ID NO: 369 - protein, 9 aa, BK polyomavirus
ECWIPDPSR

SEQ ID NO: 370 - protein, 9 aa, BK polyomavirus
CWIPDPSRN

SEQ ID NO: 371 - protein, 9 aa, BK polyomavirus
WIPDPSRNE

SEQ ID NO: 372 - protein, 9 aa, BK polyomavirus
IPDPSRNEN

SEQ ID NO: 373 - protein, 362 aa, BK polyomavirus
MAPTKRKGECPGAAPKKPKEPVQVPKLLIKGGVEVLEVKTGVDAITEVECFLNPEMGDPDENLRGFSLKLSAENDFSSDSPERKMLPCYSTARIPLPNLNEDLTCGNLLMWEAVTVQTEVIGITSMLNLHAGSQKVHEHGGGKPIQGSNFHFFAVGGEPLEMQGVLMNYRSKYPDGTITPKNPTAQSQVMNTDHKAYLDKNNAYPVECWVPDPSRNENARYFGTFTGGENVPPVLHVTNTATTVLLDEQGVGPLCKADSLYVSAADICGLFTNSSGTQQWRGLARYFKIRLRKRSVKNPYPISFLLSDLINRRTQRVDGQPMYGMESQVEEVRVFDGTERLPGDPDMIRYIDKQGQLQTKML

SEQ ID NO: 374 - protein, 9 aa, Homo sapiens
ATDFKFAMY

SEQ ID NO: 375 - protein, 9 aa, Homo sapiens
IPSYKKLIM

SEQ ID NO: 376 - protein, 9 aa, Homo sapiens
SLFRAVITK

SEQ ID NO: 377 - protein, 9 aa, Homo sapiens
IADMGHLKY